(19) United States
(12) Patent Application Publication
(10) Pub. No.: US 2010/0160386 A1
(43) Pub. Date: Jun. 24, 2010

(54) 4-PHENOXY-NICOTINE ACID DERIVATIVES AND USE THEREOF AS PPAR-MODULATORS

(75) Inventors: Heinrich Meier, Wuppertal (DE); Peter Kolkhof, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT

(21) Appl. No.: 12/440,724

(22) PCT Filed: Aug. 30, 2007
(86) PCT No.: PCT/EP07/07573

§ 371 Date: Jan. 6, 2010

(30) Foreign Application Priority Data
Sep. 12, 2006 (DE) 102006043519.2

(51) Int. Cl. A61K 31/4418; C07D 213/80; C07D 213/803; A61P 3/06; A61P 9/10; A61P 9/04

(52) U.S. Cl. 514/350; 546/298

(57) ABSTRACT

The present invention relates to novel 4-phenoxy-6-phenyl- and 4-phenoxy-6-pyridylnicotinic acid derivatives, to processes for their preparation, to their use for the treatment and/or prophylaxis of diseases and to their use for producing medicaments for the treatment and/or prophylaxis of diseases, preferably for the treatment and/or prophylaxis of cardiovascular disorders, especially of dyslipidemias, arteriosclerosis and heart failure.

The present application relates to novel 4-phenoxy-6-phenyl- and 4-phenoxy-6-pyridylnicotinic acid derivatives, to processes for their preparation, to their use for the treatment and/or prophylaxis of diseases and to their use for producing medicaments for the treatment and/or prophylaxis of diseases, preferably for the treatment and/or prophylaxis of cardiovascular disorders, especially of dyslipidemias, arteriosclerosis and heart failure.

In spite of many therapeutic successes, cardiovascular disorders remain a serious public health problem. While treatment with statins by inhibiting HMG-CoA reductase very successfully lower both the plasma concentration of LDL cholesterol (LDL-C) and the mortality of patients at risk, there is currently a lack of convincing treatment strategies for the therapy of patients with unfavorable HDL-C/LDL-C ratio or with hypertriglyceridemia.

Apart from niacin, fibrates are to date the only therapy option for patients of these risk groups. They lower elevated triglycerides by 20-50%, lower LDL-C by 10-15%, alter the LDL particle size of atherogenic low-density LDL to normal-density and less dense atherogenic LDL and increase the HDL concentrations by 10-15%.

Fibrates act as weak agonsists of the peroxisome proliferator-activated receptor (PPAR)-alpha (Nature 1990, 347, 645-50). PPAR-alpha is a nuclear receptor which regulates the expression of target genes by binding to DNA sequences in the promoter region of these genes [also known as PPAR Response Elements (PPREs)]. PPREs have been identified in a series of genes which code for proteins which regulate lipid metabolism. PPAR-alpha is expressed to a high degree in the liver and its activation leads to effects including lowered VLDL production/secretion and reduced apolipoprotein CIII (ApoCIII) synthesis. In contrast, the synthesis of apolipoprotein A1 (ApoA1) is enhanced.

One disadvantage of fibrates approved to date is their only weak interaction with the receptor (EC₅₀ in the μM range), which leads in turn to the above-described relatively minor pharmacological effects.

It was an object of the present invention to provide novel compounds which can be used as PPAR-alpha modulators for the treatment and/or prophylaxis especially of cardiovascular disorders.

WO 95/07890, WO 95/07891 and WO 95/07892 disclose substituted pyridine derivatives as pesticides and fungicides. WO 02/30358 claims various heteroaromatic compounds as modulators of the CCR4 chemokine receptor function for the treatment of allergic disorders. Variously substituted 2-arylpyridines are described in US 2003/0152520 as CRF receptor modulators for the treatment of states of anxiety and depression.

The present invention provides compounds of the general formula (I)

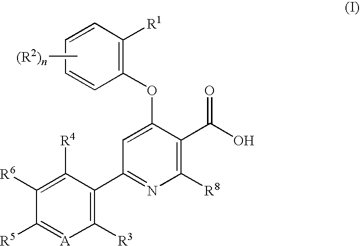

in which
R¹ is halogen, cyano or (C₁-C₄)-alkyl,
R² is a substituent selected from the group of halogen, cyano, (C₁-C₆)-alkoxy and —NR⁹—C(═O)—R¹⁰, in which alkyl and alkoxy may in turn be substituted by hydroxyl, (C₁-C₄)-alkoxy, amino, mono-(C₁-C₄)-alkylamino or di-(C₁-C₄)-alkylamino, or up to pentasubstituted by fluorine, and
   R⁹ is hydrogen or (C₁-C₆)-alkyl
   and
   R¹⁰ is hydrogen, (C₁-C₆)-alkyl or (C₁-C₆)-alkoxy,
n is 0, 1, 2 or 3,
   where, in the case that the substituent R² occurs more than once, its definitions may be identical or different,
A is N or C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, (C₁-C₄)-alkyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to pentasubstituted by fluorine,
and
R⁸ is hydrogen, methyl or trifluoromethyl,
   and the salts, solvates and solvates of the salts thereof.

Inventive compounds are the compounds of the formula (I) and the salts, solvates and solvates of the salts thereof, the compounds, encompassed by formula (I), of the formulae mentioned below and the salts, solvates and solvates of the salts thereof, and also the compounds which are encompassed by the formula (I) and are cited below as working examples and the salts, solvates and solvates of the salts thereof if the compounds which are encompassed by the formula (I) and are cited below are not already salts, solvates and solvates of the salts.

Depending on their structure, the inventive compounds can exist in stereoisomeric forms (enantiomers, diastereomers). Accordingly, the invention encompasses the enantiomers or diastereomers and their particular mixtures. From such mixtures of enantiomers and/or diastereomers, it is possible to isolate the stereoisomerically uniform components in a known manner.

If the inventive compounds can occur in tautomeric forms, the present invention encompasses all tautomeric forms.

In the context of the present invention, preferred salts are physiologically acceptable salts of the inventive compounds. The invention also comprises salts which themselves are unsuitable for pharmaceutical applications, but which can be used, for example, for isolating or purifying the inventive compounds.

Physiologically acceptable salts of the inventive compounds include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of customary bases, such as, by way of example and with preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and with preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the inventive compounds which, in the solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the inventive compounds. The term “prodrugs” includes compounds which may themselves be biologically active or inactive but which, during their time of residence in the body, are converted into inventive compounds (for example metabolically or hydrolytically).

In particular, the present invention also encompasses hydrolyzable ester derivatives of the carboxylic acids of the formula (I). This is understood to mean esters which can be hydrolyzed to the free carboxylic acids in physiological media and especially in vivo by an enzymatic or chemical route. Preferred esters of this kind are straight-chain or branched (C₁-C₆)-alkyl esters in which the alkyl group may be substituted by hydroxyl, (C₁-C₄)-alkoxy, amino, mono-(C₁-C₄)-alkylamino and/or di-(C₁-C₄)-alkylamino. Particular preference is given to the methyl or ethyl esters of the compounds of the formula (I).

In the context of the present invention, unless specified otherwise, the substituents are each defined as follows:

In the context of the invention, (C₁-C₆)-alkyl and (C₁-C₄)-alkyl are each a straight-chain or branched alkyl radical having from 1 to 6 and from 1 to 4 carbon atoms respectively. Preference is given to a straight-chain or branched alkyl radical having from 1 to 4 carbon atoms. Preferred examples include: methyl, ethyl, n-propyl, isopropyl, n-butyl, isobutyl, sec-butyl, tert-butyl, 1-ethylpropyl, n-pentyl, isopentyl and n-hexyl.

In the context of the invention, (C₁-C₆)-alkoxy and (C₁-C₄)-alkoxy are each a straight-chain or branched alkoxy radical having from 1 to 6 and from 1 to 4 carbon atoms respectively. Preference is given to a straight-chain or branched alkoxy radical having from 1 to 4 carbon atoms. Preferred examples include: methoxy, ethoxy, n-propoxy, isopropoxy, n-butoxy, tert-butoxy, n-pentoxy and n-hexoxy.

In the context of the invention, mono-(C₁-C₄)-alkylamino is an amino group having a straight-chain or branched alkyl substituent having from 1 to 4 carbon atoms. Preferred examples include: methyl-amino, ethylamino, n-propylamino, isopropylamino, n-butylamino and tert-butylamino.

In the context of the invention, di-(C₁-C₄)-alkylamino is an amino group having two identical or different straight-chain or branched alkyl substituents which each have from 1 to 4 carbon atoms. Preferred examples include: N,N-dimethylamino, N,N-diethylamino, N-ethyl-N-methylamino, N-methyl-N-n-propylamino, N-isopropyl-N-methylamino, N,N-diisopropylamino, N-n-butyl-N-methylamino and N-tert-butyl-N-methylamino.

In the context of the invention, halogen includes fluorine, chlorine, bromine and iodine. Preference is given to chlorine or fluorine.

When radicals in the inventive compounds are substituted, the radicals may, unless specified otherwise, be mono- or polysubstituted. In the context of the present invention, the definitions of radicals which occur more than once are independent of one another. Substitution with one, two or three identical or different substituents is preferred. Very particular preference is given to substitution by one substituent.

In the context of the present invention, preference is given to compounds of the formula (I) in which
R¹ is fluorine, chlorine, bromine, cyano or (C₁-C₄)-alkyl,
R² is a substituent selected from the group of fluorine, chlorine, bromine, cyano, (C₁-C₄)-alkyl and (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
n is 0, 1 or 2,
   where, in the case that the substituent R² occurs more than once, its definitions may be the same or different,
A is N or C—R⁷,
R³ is hydrogen or fluorine,
R⁴ is hydrogen, fluorine or methyl,
R⁵ is hydrogen, fluorine, chlorine, cyano, trifluoromethyl, trifluoromethoxy or (C₁-C₄)-alkoxy,
R⁶ and R⁷ are the same or different and are each independently hydrogen, fluorine, chlorine, bromine, cyano, (C₁-C₄)-alkyl or (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
and
R⁸ is hydrogen, methyl or trifluoromethyl,
   and the salts, solvates and solvates of the salts thereof.

Of particular significance in the context of the present invention are compounds of the formula (I) in which
R¹ is fluorine, chlorine, bromine, cyano or methyl,
   and the salts, solvates and solvates of the salts thereof.

Equally of particular significance in the context of the present invention are compounds of the formula (I) in which
R³ and R⁴ are each independently hydrogen or fluorine,
   and the salts, solvates and solvates of the salts thereof.

Equally of particular significance in the context of the present invention are compounds of the formula (I) in which
R⁵ is hydrogen, fluorine, chlorine, methyl or trifluoromethyl,
   and the salts, solvates and solvates of the salts thereof.

In the context of the present invention, particular preference is given to compounds of the formula (I) in which
R¹ is fluorine, chlorine, bromine, cyano or methyl,
R² is a substituent selected from the group of fluorine, chlorine, bromine, cyano, (C₁-C₄)-alkyl, trifluoromethyl, (C₁-C₄)-alkoxy and trifluoromethoxy,
n is 0, 1 or 2,
   where, in the case that the substituent R² occurs more than once, its definitions may be the same or different,
A is C—R⁷,
R³ is hydrogen,
R⁴ is hydrogen or fluorine,
R⁵ is hydrogen, fluorine, chlorine, methyl or trifluoromethyl,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
and
R⁸ is hydrogen,
   and the salts, solvates and solvates of the salts thereof.

The radical definitions specified individually in the particular combinations or preferred combinations of radicals are, irrespective of the particular combinations of the radicals specified, also replaced as desired by radical definitions of other combinations.

Very particular preference is given to combinations of two or more of the abovementioned preferred ranges.

The invention further provides a process for preparing the inventive compounds of the formula (I), characterized in that a compound of the formula (II)

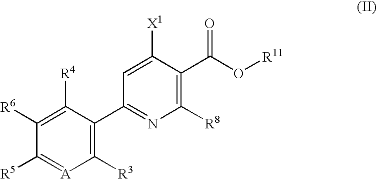

in which A, R³, R⁴, R⁵, R⁶ and R⁸ are each as defined above,
X¹ is a suitable leaving group, for example halogen, especially chlorine,
and
R¹¹ is (C₁-C₄)-alkyl,
   in an inert solvent in the presence of a base, is reacted with a compound of the formula (III)

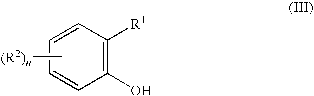

in which R¹, R² and n are each as defined above
to give compounds of the formula (IV)

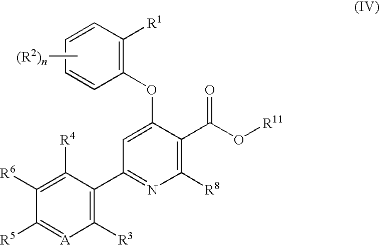

in which A, R¹, R², R³, R⁴, R⁵, R⁶, R⁸, R¹¹ and n are defined as specified above,
and these compounds are then converted to the carboxylic acids of the formula (I) by basic or acidic hydrolysis
and the compounds of the formula (I) are optionally reacted with the corresponding (i) solvents and/or (ii) bases or acids to give their solvates, salts and/or solvates of the salts.

Inert solvents of the process step (II)+(III)→(IV) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as dimethylformamide, dimethyl sulfoxide, N,N′-dimethylpropyleneurea (DMPU), N-methyl-pyrrolidinone (NMP), pyridine, acetone, 2-butanone or acetonitrile. It is equally possible to use mixtures of the solvents mentioned. Preference is given to using dimethylformamide.

Suitable bases for the process step (II)+(III)→(IV) are customary inorganic bases. These include especially alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or cesium carbonate, or alkali metal hydrides such as sodium hydride or potassium hydride. Preference is given to potassium carbonate.

The base is used here in an amount of from 1 to 5 mol, preferably in an amount of from 1.2 to 3 mol, based on 1 mol of the compound of the formula (III). The reaction is effected generally within a temperature range from 0° C. to +150° C., preferably at from +20° C. to +100° C. The reaction can be performed at standard, elevated or reduced pressure (for example from 0.5 to 5 bar). In general, standard pressure is employed.

The hydrolysis of the carboxylic ester in process step (IV)→(I) is effected by customary methods by treating the esters with acids or bases in inert solvents, and the salts formed initially in the latter case are converted to the free carboxylic acids by subsequent treatment with acids. In the case of the tert-butyl esters, the ester cleavage is effected preferably with acids.

Suitable inert solvents for the hydrolysis of the carboxylic esters are water or the organic solvents customary for an ester cleavage. These include especially alcohols such as methanol, ethanol, n-propanol, isopropanol, n-butanol or tert-butanol, ethers such as diethyl ether, tetrahydrofuran, dioxane or glycol dimethyl ether, or other solvents such as acetone, acetonitrile, dichloromethane, dimethylformamide or dimethyl sulfoxide. It is equally possible to use mixtures of the solvents mentioned. In the case of a basic ester hydrolysis, preference is given to using mixtures of water with dioxane, tetrahydrofuran, methanol and/or ethanol. In the case of the reaction with trifluoroacetic acid, preference is given to using dichloromethane, and, in the case of the reaction with hydrogen chloride, preference is given to using tetrahydrofuran, diethyl ether, dioxane or water.

Suitable bases for the ester hydrolysis are the customary inorganic bases. These include especially alkali metal or alkaline earth metal hydroxides, for example sodium hydroxide, lithium hydroxide, potassium hydroxide or barium hydroxide, or alkali metal or alkaline earth metal carbonates such as sodium carbonate, potassium carbonate or calcium carbonate. Preference is given to using sodium hydroxide or lithium hydroxide.

Suitable acids for the ester cleavage are generally sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid or mixtures thereof, optionally with addition of water. Preference is given to hydrogen chloride or trifluoroacetic acid in the case of the tert-butyl esters, and hydrochloric acid in the case of the methyl esters.

The esters are cleaved generally within a temperature range from 0° C. to +100° C., preferably at from 0° C. to +50° C. The reaction can be performed at standard, elevated or reduced pressure (for example from 0.5 to 5 bar). In general, standard pressure is employed.

Optionally, the ester hydrolysis (IV)→(I) can also advantageously be effected directly in the reaction mixture of the preparation of compound (IV), such that it is possible to dispense with an intermediate isolation of the compound (IV).

The compounds of the formula (II) can be prepared by

### [A] Coupling a Compound of the Formula (V)

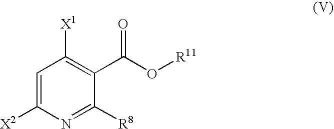

in which R⁸ and R¹¹ are each as defined above and
X¹ and X² are the same or different and are each a suitable leaving group, for example halogen, especially chlorine,
in an inert solvent in the presence of a suitable transition metal catalyst to a compound of the formula (VI)

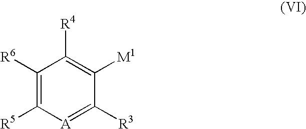

in which A, R³, R⁴, R⁵ and R⁶ are each as defined above and
M¹ is the —ZnHal or —MgHal group, in which
   Hal is halogen, especially chlorine, bromine or iodine,
      or, in the case that R⁸ in formula (II) is hydrogen,

### [B] Reacting a Compound of the Formula (VII)

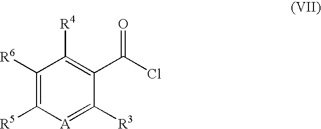

in which A, R³, R⁴, R⁵ and R⁶ are each as defined above
first in an inert solvent in the presence of a base with a compound of the formula (VIII)

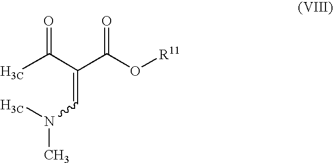

in which R¹¹ is as defined above,
and then with an ammonia source, for example ammonium chloride, to give a compound of the formula (IX)

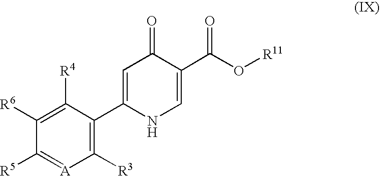

in which A, R³, R⁴, R⁵, R⁶ and R¹¹ are each as defined above,
and then converting this with the aid of a suitable chlorinating agent, for example phosphorus oxychloride, to a compound of the formula (II-A)

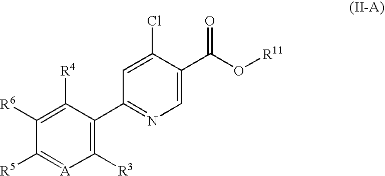

in which A, R³, R⁴, R⁵, R⁶ and R¹¹ are each as defined above,
[for the transformation (VII)→(IX), see, for example, S. W. McCombie et al., J. Org. Chem. 56, 4963-4967 (1991)].

The compounds of the formulae (III), (V), (VI), (VII) and (VIII) are commercially available, known from the literature or can be prepared in analogy to literature processes. In the case of an organozinc compound of the formula (VI) [M¹=ZnHal], it can optionally also be obtained in situ from the corresponding Grignard compound [M¹=MgHal] and a zinc halide [cf., for example, Fu et al., J. Am. Chem. Soc. 123, 2719-2724 (2001)].

Transition metal catalysts and catalyst ligands for the coupling reactions (V)+(VI)→(II) are known from the literature [cf., for example, J. Hassan et al., Chem. Rev. 102, 1359-1469 (2002)] and commercially available. In the case of organozinc compounds [M¹=ZnHal in (VI)], preference is given to using tetrakis(triphenylphosphine)palladium(0) as the catalyst.

The reactions (V)+(VI)→(II) are effected generally within a temperature range of from −20° C. to +120° C., preferably at from 0° C. to +60° C. The reactions can be performed at standard, elevated or reduced pressure (for example from 0.5 to 5 bar). In general, standard pressure is employed.

The invention further provides a process for preparing the inventive compounds of the formula (I), characterized in that a compound of the formula (V)

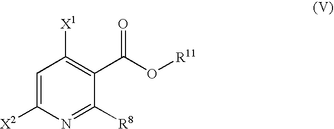

in which R⁸ is as defined above,
R¹¹ is (C₁-C₄)-alkyl
and
   X¹ and X² are the same or different and are each a suitable leaving group, for example halogen, especially chlorine,
   is first reacted in an inert solvent in the presence of a base with a compound of the formula (III)

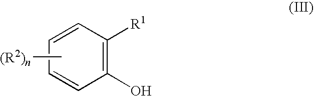

in which R¹, R² and n are each as defined above
to give compounds of the formula (X)

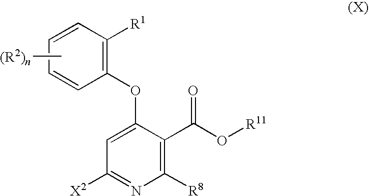

in which R¹, R², R⁸, R¹¹, X² and n are each as defined above,
then this is coupled in an inert solvent in the presence of a suitable transition metal catalyst and of a base to a compound of the formula (VIa)

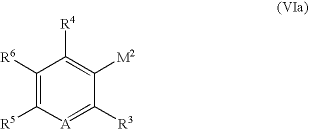

in which A, R³, R⁴, R⁵ and R⁶ are each as defined above and
M² is the —B(OH)₂, —ZnHal or —MgHal group, in which
   Hal is halogen, especially chlorine, bromine or iodine,
      to give compounds of the formula (IV)

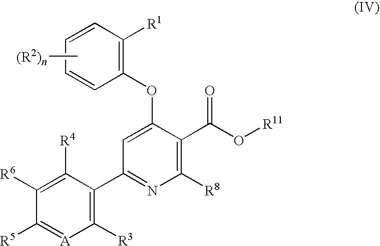

in which A, R¹, R², R³, R⁴, R⁵, R⁶, R⁸, R¹¹ and n are each as defined above,
and then converted by basic or acidic hydrolysis to the carboxylic acids of the formula (I),
and the compounds of the formula (I) are optionally reacted with the corresponding (i) solvents and/or (ii) bases or acids to give their solvates, salts and/or solvates of the salts.

For the process step (V)+(III)→(X), the reaction parameters described above for the reaction (II)+(III)→(IV), such as solvents, bases and temperature, find use in an analogous manner.

Inert solvents for the boronic acid coupling [“Suzuki coupling”] in the process step (X)+(VIa)→(IV) [M²=B(OH)₂] are, for example, alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as dimethylformamide, dimethyl sulfoxide, N,N′-dimethylpropyleneurea (DMPU), N-methylpyrrolidone (NMP), pyridine, acetonitrile or else water. It is equally possible to use mixtures of the solvents mentioned. Preference is given to using dimethylformamide or dioxane.

Suitable auxiliary bases for this reaction are customary inorganic bases. They include especially alkali metal hydroxides, for example lithium hydroxide, sodium hydroxide or potassium hydroxide, alkali metal hydrogencarbonates such as sodium hydrogencarbonate or potassium hydrogencarbonate, alkali metal or alkaline earth metal carbonates such as lithium carbonate, sodium carbonate, potassium carbonate, calcium carbonate or cesium carbonate, or alkali metal hydrogenphosphates such as disodium hydrogenphosphate or dipotassium hydrogenphosphate. Preference is given to using sodium carbonate or potassium carbonate.

In the coupling with organozinc or organomagnesium compounds (VIa) [M²=ZnHal or MgHal], suitable inert solvents are especially ethers such as diethyl ether, di-n-butyl ether, tetrahydrofuran or glycol dimethyl ether, or hydrocarbons such as benzene, toluene, hexane or cyclohexane. Preference is given to using tetrahydrofuran.

Transition metal catalysts and catalyst ligands for the coupling reaction (X)+(VIa)→(IV) are known from the literature [cf., for example, J. Hassan et al., Chem. Rev. 102, 1359-1469 (2202)] and are commercially available. Preference is given to using palladium or nickel catalysts. For a boronic acid coupling [M²=B(OH)₂ in (VIa)], for example, palladium(II) acetate, tetrakis-(triphenylphosphine)palladium(0), bis(triphenylphosphine)palladium(II) chloride, bis(acetonitrile)-palladium(II) chloride or [1,1′-bis(diphenylphosphino)ferrocene]dichloropalladium(II)-dichloro-methane complex are suitable, optionally in the presence of an additional catalyst ligand such as tris(o-tolyl)phosphine. Preference is given to using bis(triphenylphosphine)palladium(II) chloride. In a coupling with organozinc compounds [M²=ZnHal in (VIa)], preference is given to using tetrakis(triphenylphosphine)palladium(0) as the catalyst.

The coupling reactions (X)+(VIa)→(IV) are effected generally within a temperature range from −20° C. to +150° C., preferably at from 0° C. to +100° C. The reactions can be performed at standard, at elevated or at reduced pressure (for example from 0.5 to 5 bar). In general, standard pressure is employed.

The compounds of the formula (VIa) are commercially available, are known from the literature or can be obtained in analogy to literature processes. In the case of an organozinc compound of the formula (VIa) [M²=ZnHal], it can optionally also be obtained in situ from the corresponding Grignard compound [M²=MgHal] and a zinc halide [cf., for example, Fu et al., J. Am. Chem. Soc. 123, 2719-2724 (2001)].

The preparation of the inventive compounds can be illustrated by the following synthesis schemes:

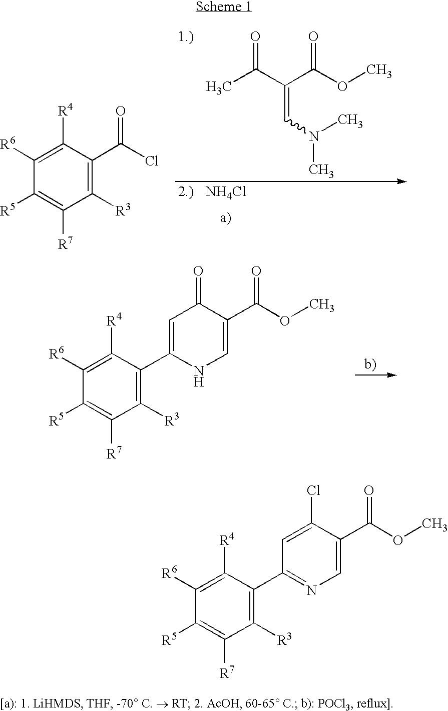

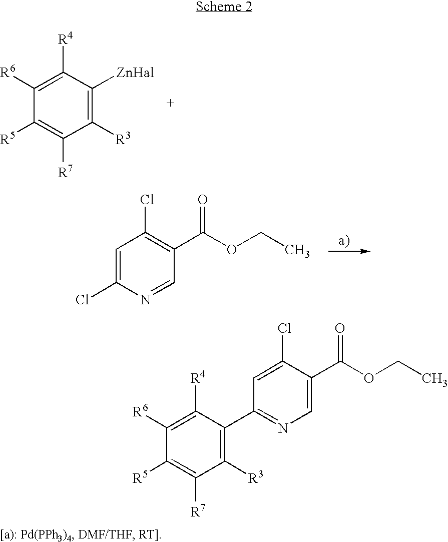

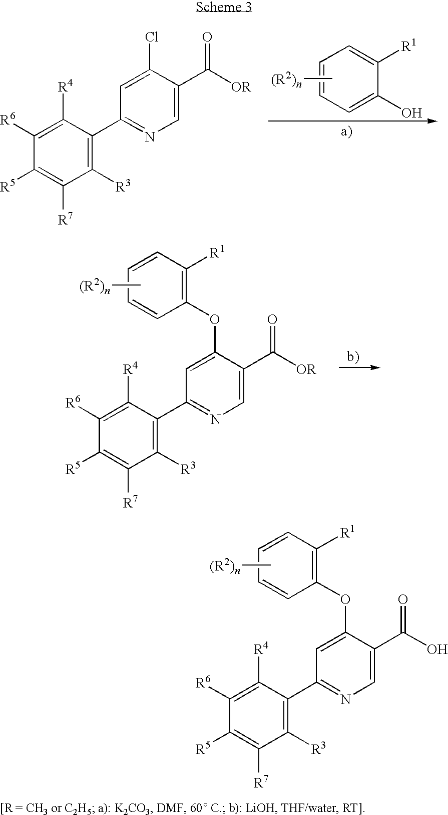

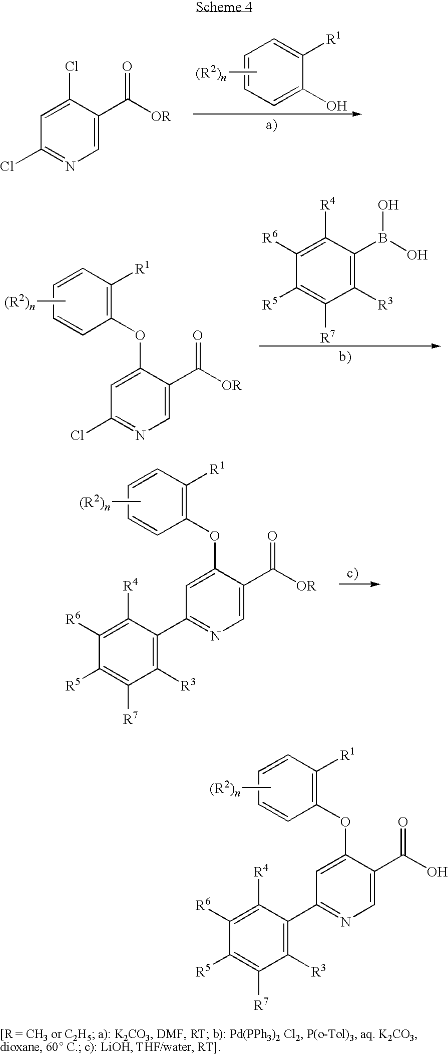

The inventive compounds have valuable pharmacological properties and can be used for the prevention and treatment of disorders in humans and animals.

The inventive compounds are highly active PPAR-alpha modulators and are suitable as such especially for the primary and/or secondary prevention and treatment of cardiovascular disorders which are caused by disruptions in the fatty acid and glucose metabolism. Such disorders include dyslipidemias (hypercholesterolemia, hypertriglyceridemia, elevated concentrations of the postprandial plasma triglycerides, hypoalphalipoproteinemia, combined hyperlipidemias), arteriosclerosis and metabolic disorders (metabolic syndrome, hyperglycemia, insulin-dependent diabetes, non-insulin-dependent diabetes, gestation diabetes, hyperinsulinemia, insulin resistance, glucose intolerance, adiposity and diabetic late complications such as retinopathy, nephropathy and neuropathy).

As highly active PPAR-alpha modulators, the inventive compounds are suitable especially also for the primary and/or secondary prevention and treatment of heart failure.

In the context of the present invention, the term “heart failure” also encompasses more specific or related disease forms such as right heart failure, left heart failure, global failure, ischemic cardiomyopathy, dilatative cardiomyopathy, congenital heart defects, heart valve defects, heart failure in the event of heart valve defects, mitral valve stenosis, mitral valve failure, aortic valve stenosis, aortic valve failure, tricuspidal stenosis, tricuspidal failure, pulmonary valve stenosis, pulmonary valve failure, combined heart valve defects, heart muscle inflammation (myocarditis), chronic myocarditis, acute myocarditis, viral myocarditis, diabetic heart failure, alcohol-toxic cardiomyopathy, cardiac storage disorders, diastolic heart failure and systolic heart failure.

Further independent risk factors for cardiovascular disorders which can be treated by the inventive compounds are hypertension, ischemia, myocardial infarction, angina pectoris, heart muscle weakness, restenosis, pulmonary hypertension, increased levels of fibrinogen and of low-density LDL and elevated concentrations of plasminogen activator inhibitor 1 (PAI-1).

Furthermore, the inventive compounds may also be used for the treatment and/or prevention of micro- and macrovascular damage (vasculitis), reperfusion damage, arterial and venous thromboses, edemas, cancers (skin cancer, liposarcomas, carcinomas of the gastrointestinal tract, of the liver, pancreas, lung, kidney, ureter, prostate and of the genital tract), of disorders of the central nervous system and neurodegenerative disorders (stroke, Alzheimer's disease, Parkinson's disease, dementia, epilepsy, depression, multiple sclerosis), of inflammatory disorders, immune disorders (Crohn's disease, ulcerative colitis, lupus erythematosus, rheumatoid arthritis, asthma), kidney disorders (glomerulonephritis), thyroid disorders (hyperthyreosis), disorders of the pancreas (pancreatitis), liver fibrosis, skin disorders, (psoriasis, acne, eczema, neurodermitis, dermatitis, keratitis, scar formation, wart formation, chillblains), viral disorders (HPV, HCMV, HIV), cachexia, osteoporosis, gout, incontinence, and for wound healing and angiogenesis.

The efficacy of the inventive compounds can be tested, for example, in vitro by the transactivation assay described in the example part.

The efficacy of the inventive compounds in vivo can be tested, for example, by the studies described in the example part.

The present invention further provides for the use of the inventive compounds for the treatment and/or prevention of disorders, especially of the aforementioned disorders.

The present invention further provides for the use of the inventive compounds for producing a medicament for the treatment and/or prevention of disorders, especially of the aforementioned disorders.

The present invention further provides a process for the treatment and/or prevention of disorders, especially of the aforementioned disorders, using an effective amount of at least one of the inventive compounds.

The inventive compounds may be used alone or, if required, in combination with other active ingredients. The present invention further provides medicaments comprising at least one of the inventive compounds and one or more further active ingredients, especially for the treatment and/or prevention of the aforementioned disorders.

Suitable active ingredients for combinations include, by way of example and with preference: substances which modify lipid metabolism, antidiabetics, hypotensives, perfusion-enhancing and/or antithrombotic agents, and also antioxidants, chemokine receptor antagonists, p38-kinase inhibitors, NPY agonists, orexin agonists, anorectics, PAF-AH inhibitors, antiphlogistics (COX inhibitors, LTB₄-receptor antagonists), analgesics (aspirin), antidepressants and other psychopharmaceuticals.

The present invention provides especially combinations comprising at least one of the inventive compounds and at least one lipid metabolism-modifying active ingredient, an antidiabetic, an active hypotensive ingredient and/or an antithrombotic agent.

The inventive compounds can preferably be combined with one or more
lipid metabolism-modifying active ingredients, by way of example and with preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbers, bile acid reabsorption inhibitors, MTP inhibitors, lipase inhibitors, LpL activators, fibrates, niacin, CETP inhibitors, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavengers,
antidiabetics mentioned in the Rote Liste 2004/II, chapter 12, and also, by way of example and with preference, those from the group of the sulfonylureas, biguanides, meglitinide derivatives, glucosidase inhibitors, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861,
active hypotensive ingredients, by way of example and with preference from the group of the calcium antagonists, angiotensin AII antagonists, ACE inhibitors, beta-receptor blockers, alpha-receptor blockers, ECE inhibitors and the vasopeptidase inhibitors;
antithrombotic agents, by way of example and with preference from the group of the platelet aggregation inhibitors or the anticoagulants;
diuretics;
aldosterone and mineral corticoid receptor antagonists;
vasopressin receptor antagonists;
organic nitrates and NO donors;
positive-inotropically active ingredients;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, in particular PDE 5 inhibitors such as sildenafil, vardenafil and tadalafil, and PDE 3 inhibitors such as milrinone;
natriuretic peptides such as for example “atrial natriuretic peptide” (ANP, anaritide), “B-type natriuretic peptide” or “brain natriuretic peptide” (BNP, nesiritide), “C-type natriuretic peptide” (CNP) and urodilatin;
calcium sensitizers, by way of example and with preference levosimendan;
potassium supplements;
NO-independent but heme-dependent stimulators of guanylate cyclase, especially the compounds described in WO 00/06568, WO 00/06569, WO 02/42301 and WO 03/095451;
NO- and heme-independent activators of guanylate cyclase, especially the compounds described in WO 01/19355, WO 01/19776, WO 01/19778, WO 01/19780, WO 02/070462 and WO 02/070510;
inhibitors of human neutrophil elastase (HNE), for example sivelestat or DX-890 (reltran);
compounds inhibiting the signal transduction cascade, for example tyrosine kinase inhibitors, in particular sorafenib, imatinib, gefitinib and erlotinib; and/or
compounds influencing the energy metabolism of the heart, for example etomoxir, dichloroacetate, ranolazine or trimetazidine.

Lipid metabolism-modifying active ingredients are preferably understood to mean compounds from the group of the HMG-CoA reductase inhibitors, squalene synthesis inhibitors, ACAT inhibitors, cholesterol absorption inhibitor, MTP inhibitors, lipase inhibitors, thyroid hormones and/or thyroid mimetics, niacin receptor agonists, CETP inhibitors, PPAR-gamma agonists, PPAR-delta agonists, polymeric bile acid adsorbers, bile acid reabsorption inhibitors, antioxidants/radical scavengers and also the cannabinoid receptor 1 antagonists.

In a preferred embodiment of the invention, the inventive compounds are administered in combination with an HMG-CoA reductase inhibitor from the class of the statins, by way of example and with preference lovastatin, simvastatin, pravastatin, fluvastatin, atorvastatin, rosuvastatin, cerivastatin or pitavastatin.

In a preferred embodiment of the invention, the inventive compounds are administered in combination with a squalene synthesis inhibitor, by way of example and with preference BMS-188494 or TAK-475.

In a preferred embodiment of the invention, the inventive compounds are administered in combination with an ACAT inhibitor, by way of example and with preference melinamide, pactimibe, eflucimibe or SMP-797.

In a preferred embodiment of the invention, the compounds according to the invenetion are administered in combination with a cholesterol absorption inhibitor, by way of example and with preference ezetimibe, tiqueside or pamaqueside.

In a preferred embodiment of the invention, the inventive compounds are administered in combination with an MTP inhibitor, by way of example and with preference implitapide or JTT-130.

In a preferred embodiment of the invention, the inventive compounds are administered in combination with a lipase inhibitor, by way of example and with preference orlistat.

In a preferred embodiment of the invention, the inventive compounds are administered in combination with a thyroid hormone and/or thyroid mimetic, by way of example and with preference D-thyroxine or 3,5,3′-triiodothyronine (T3).

In a preferred embodiment of the invention, the inventive compounds are administered in combination with an agonist of the niacin receptor, by way of example and with preference niacin, acipimox, acifran or radecol.

In a preferred embodiment of the invention, the inventive compounds are administered in combination with a CETP inhibitor, by way of example and with preference torcetrapib, JTT-705 or CETP vaccine (Avant).

In a preferred embodiment of the invention, the inventive compounds are administered in combination with a PPAR-gamma agonist, by way of example and with preference pioglitazone or rosiglitazone.

In a preferred embodiment of the invention, the inventive compounds are administered in combination with a PPAR-delta agonist, by way of example and with preference GW-501516.

In a preferred embodiment of the invention, the inventive compounds are administered in combination with a polymeric bile acid adsorber, by way of example and with preference cholestyramine, colestipol, colesolvam, CholestaGel or colestimide.

In a preferred embodiment of the invention, the inventive compounds are administered in combination with a bile acid reabsorption inhibitor, by way of example and with preference ASBT (=IBAT) inhibitors, such as, for example, AZD-7806, S-8921, AK-105, BARI-1741, SC-435 or SC-635.

In a preferred embodiment of the invention, the inventive compounds are administered in combination with a antioxidant/radical scavenger, by way of example and with preference probucol, AGI-1067, BO-653 or AEOL-10150.

In a preferred embodiment of the invention, the inventive compounds are administered in combination with a cannabinoid receptor 1 antagonist, by way of example and with preference rimonabant or SR-147778.

Antidiabetics are preferably understood to mean insulin and insulin derivatives, and also orally active hypoglycemic acid compounds. Here, insulin and insulin derivatives include both insulins of animal, human or biotechnological origin and also mixtures thereof. The orally active hypoglycemic active ingredients preferably include sulfonylureas, biguanides, meglitinide derivatives, glucosidase inhibitors and PPAR-gamma agonists.

In a preferred embodiment of the invention, the inventive compounds are administered in combination with insulin.

In a preferred embodiment of the invention, the inventive compounds are administered in combination with a sulfonylurea, by way of example and with preference tolbutamide, glibenclamide, glimepiride, glipizide or gliclazide.

In a preferred embodiment of the invention, the inventive compounds are administered in combination with a biguanide, by way of example and with preference metformin

In a preferred embodiment of the invention, the inventive compounds are administered in combination with a meglitinide derivative, by way of example and with preference repaglinide or nateglinide.

In a preferred embodiment of the invention, the inventive compounds are administered in combination with a glucosidase inhibitor, by way of example and with preference miglitol or acarbose.

In a preferred embodiment of the invention, the inventive compounds are administered in combination with a PPAR-gamma agonist, for example from the class of the thiazolidinediones, by way of example and with preference pioglitazone or rosiglitazone.

The hypotensive agents are preferably understood to mean compounds from the group of the calcium antagonists, angiotensin AII antagonists, ACE inhibitors, beta-receptor blockers, alpha-receptor blockers and of the diuretics.

In a preferred embodiment of the invention, the inventive compounds are administered in combination with a diuretic, by way of example and with preference a loop diuretic such as furosemide, bumetanide or torsemide, or a thiazide or thiazide-like diuretic such as chlorothiazide or hydrochlorothiazide.

In a preferred embodiment of the invention, the inventive compounds are administered in combination with an aldosterone or mineral corticoid receptor antagonist, by way of example and with preference spironolactone or eplerenone.

In a preferred embodiment of the invention, the inventive compounds are administered in combination with a vasopressin receptor antagonist, by way of example and with preference conivaptan, tolvaptan, lixivaptan or SR-121463.

In a preferred embodiment of the invention, the inventive compounds are administered in combination with an organic nitrate or NO donor, by way of example and with preference sodium nitroprusside, nitroglycerine, isosorbide mononitrate, isosorbide dinitrate, molsidomine or SIN-1, or in combination with inhalative NO.

In a preferred embodiment of the invention, the inventive compounds are administered in combination with a positively-inotropically active compound, by way of example and with preference cardiac glycosides (digoxin), beta-adrenergic and dopaminergic agonists such as isoproterenol, adrenalin, noradrenalin, dopamine or dobutamine.

In a preferred embodiment of the invention, the inventive compounds are administered in combination with a calcium antagonist, by way of example and with preference nifedipine, amlodipine, verapamil or diltiazem.

In a preferred embodiment of the invention, the inventive compounds are administered in combination with an angiotensin AII antagonist, by way of example and with preference losartan, valsartan, candesartan, embusartan or telmisartan.

In a preferred embodiment of the invention, the inventive compounds are administered in combination with an ACE inhibitor, by way of example and with preference enalapril, captopril, ramipril, delapril, fosinopril, quinopril, perindopril or trandopril.

In a preferred embodiment of the invention, the inventive compounds are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an alpha-receptor blocker, by way of example and with preference prazosin.

In a preferred embodiment of the invention, the inventive compounds are administered in combination with antisympathotonics, by way of example and with preference reserpine, clonidine or alpha-methyldopa, or in combination with a potassium channel agonist, by way of example and with preference minoxidil, diazoxide, dihydralazine or hydralazine.

Antithrombotics are preferably understood to mean compounds from the group of the platelet aggregation inhibitors or of the anticoagulants.

In a preferred embodiment of the invention, the inventive compounds are administered in combination with a platelet aggregation inhibitor, by way of example and with preference aspirin, clopidogrel, ticlopidine or dipyridamol.

In a preferred embodiment of the invention, the inventive compounds are administered in combination with a thrombin inhibitor, by way of example and with preference ximelagatran, melagatran, bivalirudin or clexane.

In a preferred embodiment of the invention, the inventive compounds are administered in combination with a GPIIb/IIIa antagonist, by way of example and with preference tirofiban or abciximab.

In a preferred embodiment of the invention, the inventive compounds are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the inventive compounds are administered in combination with heparin or a low molecular weight (LMW) heparin derivative.

In a preferred embodiment of the invention, the inventive compounds are administered in combination with a vitamin K antagonist, by way of example and with preference coumarin.

In the context of the present invention, particular preference is given to combinations comprising at least one of the inventive compounds and one or more further active ingredients selected from the group consisting of HMG-CoA reductase inhibitors (statins), diuretics, beta-receptor blockers, organic nitrates and NO donors, ACE inhibitors, angiotensin AII antagonists, aldosterone receptor and mineralocorticoid receptor antagonists, vasopressin receptor antagonists, platelet aggregation inhibitors and anticoagulants, and to the use thereof for the treatment and/or prevention of the aforementioned disorders.

The present invention further provides medicaments which comprise at least one inventive compound, typically together with one or more inert, non-toxic, pharmaceutically suitable excipients, and the use therefore for the aforementioned purposes.

The inventive compounds can act systemically and/or locally. For this purpose, they can be administered in a suitable manner, for example orally, parenterally, pulmonally, nasally, sublingually, lingually, buccally, rectally, dermally, transdermally, conjunctivally, otically, or as an implant or stent.

For these administration routes, the inventive compounds can be administered in suitable administration forms.

Suitable for oral administration are administration forms which work in accordance with the prior art and release the inventive compounds rapidly and/or in modified form and which comprise the inventive compounds in crystalline and/or amorphicized and/or dissolved form, for example tablets (uncoated or coated tablets, for example with enteric coats or coats which dissolve in a delayed manner or are insoluble and which control the release of the inventive compounds), films/wafers or tablets which dissolve rapidly in the oral cavity, films/lyophilizates, capsules (for example hard or soft gelatin capsules), sugar-coated tablets, granules, pellets, powders, emulsions, suspensions, aerosols or solutions.

Parenteral administration may take place with avoidance of a bioabsorption step (for example intravenously, intraarterially, intracardially, intraspinally or intralumbarly), or with bioabsorption (for example intramuscularly, subcutaneously, intracutaneously, percutaneously or intraperitoneally). Administration forms suitable for parenteral administration are inter alia preparations for injection or infusion in the form of solutions, suspensions, emulsions, lyophilizates or sterile powders.

Suitable for other administration routes are, for example, medicaments suitable for inhalation (inter alia powder inhalers, nebulizers), nose drops, solutions or sprays, tablets to be administered lingually, sublingually or buccally, films/wafers or capsules, suppositories, preparations to be administered to ears or eyes, vaginal capsules, aqueous suspensions (lotions, shaking mixtures), lipophilic suspensions, ointments, creams, transdermal therapeutic systems (for example plasters), milk, pastes, foams, powders for pouring, implants or stents.

Preference is given to oral or parenteral administration, in particular to oral and intravenous administration.

The inventive compounds can be converted into the administration forms mentioned. This can be carried out in a manner known per se by mixing with inert non-toxic pharmaceutically suitable auxiliaries. These auxiliaries include inter alia carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, for example ascorbic acid), colorants (for example inorganic pigments, for example iron oxides), and flavor and/or odor corrigents.

In general, it has been found to be advantageous in the case of parenteral administration to administer amounts of about 0.001 to 1 mg/kg, preferably about 0.01 to 0.5 mg/kg of body weight to obtain effective results. In the case of oral administration, the dosage is from about 0.01 to 100 mg/kg, preferably from about 0.01 to 20 mg/kg and very particularly preferably from 0.1 to 10 mg/kg of body weight.

In spite of this, it may be necessary to deviate from the amounts mentioned, namely depending on body weight, administration route, individual response to the active compound, the type of preparation and the time or the interval at which administration takes place. Thus, in some cases it may be sufficient to administer less than the abovementioned minimum amount, whereas in other cases the upper limit mentioned has to be exceeded. In the case of the administration of relatively large amounts, it may be expedient to divide these into a plurality of individual doses which are administered over the course of the day.

The working examples below illustrate the invention. The invention is not limited to the examples.

The percentages in the tests and examples below are, unless stated otherwise, percentages by weight; parts are parts by weight. Solvent ratios, dilution ratios and concentrations of liquid/liquid solutions are in each case based on volume.

### A. EXAMPLES

### Abbreviations

AcOH acetic acid
aq. aqueous
TLC thin-layer chromatography
DCI direct chemical ionization (in MS)
DMF dimethylformamide
DMSO dimethyl sulfoxide
eq. equivalent(s)
ESI electrospray ionization (in MS)
h hour(s)
Hal halogen
HPLC high-pressure, high-performance liquid chromatography
LC-MS liquid chromatography-coupled mass spectrometry
LiHMDS lithium hexamethyldisilazide
min minute(s)
MS mass spectrometry
NMR nuclear magnetic resonance spectrometry
o-Tol ortho-tolyl
Ph phenyl
RP reverse phase (in HPLC)
RT room temperature
Rₜ retention time (in HPLC)
THF tetrahydrofuran
UV ultraviolet spectrometry
v/v volume-to-volume ratio (of a mixture)

### LC-MS and HPLC Methods:

### Method 1 (LC-MS):

Instrument type MS: Micromass ZQ; Instrument type HPLC: HP 1100 series; UV DAD; column: Phenomenex Gemini 3μ, 30 mm×3.00 mm; eluent A: 1 l water+0.5 ml 50% formic acid, eluent B: 1 l acetonitrile+0.5 ml 50% formic acid; gradient: 0.0 min 90% A →2.5 min 30% A →3.0 min 5% A →4.5 min 5% A; flow rate: 0.0 min 1 ml/min →2.5 min/3.0 min/4.5 min 2 ml/min; oven: 50° C.; UV detection: 210 nm

### Method 2 (LC-MS):

Instrument type MS: Micromass ZQ; Instrument type HPLC: Waters Alliance 2795; column: Phenomenex Synergi 2 μHydro-RP Mercury 20 mm×4 mm; eluent A: 1 l water+0.5 ml 50% formic acid, eluent B: 1 l acetonitrile+0.5 ml 50% formic acid; gradient: 0.0 min 90% A →2.5 min 30% A →3.0 min 5% A →4.5 min 5% A; flow rate: 0.0 min 1 ml/min →2.5 min/3.0 min/4.5 min 2 ml/min; oven: 50° C.; UV detection: 210 nm

### Method 3 (LC-MS):

Instrument: Micromass Quattro LCZ with HPLC Agilent series 1100; column: Phenomenex Onyx Monolithic C18, 100 mm×3 mm; eluent A: 1 l water+0.5 ml 50% formic acid, eluent B: 1 l acetonitrile+0.5 ml 50% formic acid; gradient: 0.0 min 90% A →2 min 65% A →4.5 min 5% A →6 min 5% A; flow rate: 2 ml/min; oven: 40° C.; UV detection: 208-400 nm

### Method 4 (LC-MS):

Instrument: Micromass Quattro LCZ with HPLC Agilent series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A →2.5 min 30% A →3.0 min 5% A →4.5 min 5% A; flow rate: 0.0 min 1 ml/min →2.5 min/3.0 min/4.5 min 2 ml/min; oven: 50° C.; UV detection: 208-400 nm

### Method 5 (LC-MS):

Instrument type MS: Waters ZQ; Instrument type HPLC: Waters Alliance 2795; column: Merck Chromolith RP18e, 100 mm×3 mm; eluent A: 1 l water+0.5 ml 50% formic acid, eluent B: 1 l acetonitrile+0.5 ml 50% formic acid; gradient: 0.0 min 90% A →2 min 65% A →4.5 min 5% A →6 min 5% A; flow rate: 2 ml/min; oven: 40° C.; UV detection: 210 nm

### Method 6 (LC-MS):

Instrument: Micromass Quattro LCZ with HPLC Agilent series 1100; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A →2.5 min 30% A →3.0 min 5% A →4.5 min 5% A; flow rate: 0.0 min 1 ml/min →2.5 min/3.0 min/4.5 min 2 ml/min; oven: 50° C.; UV detection: 208-400 nm

### Method 7 (LC-MS):

Instrument type MS: Micromass ZQ; Instrument type HPLC: HP 1100 series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A →2.5 min 30% A →3.0 min 5% A →4.5 min 5% A; flow rate: 0.0 min 1 ml/min →2.5 min/3.0 min/4.5 min 2 ml/min; oven: 50° C.; UV detection: 210 nm

### Method 8 (Preparative HPLC):

Instrument: Abimed Gilson Pump 305/306, Manometric Module 806; column: Grom-Sil C18 10 μm, 250 mm×30 mm; eluent: A=water, B=acetonitrile; gradient 0.0 min 10% B →3 min 10% B →30 min 95% B →42 min 95% B →42.1 min 10% B →45 min 10% B; flow rate: 50 ml/min; column temperature: RT; UV detection: 210 nm

### Method 9 (Preparative HPLC):

Instrument: Abimed Gilson Pump 305/306, Manometric Module 806; column: Grom-Sil C18 10 μm, 250 mm×30 mm; eluent: A=water, B=acetonitrile; gradient 0.0 min 30% B →3 min 30% B →30 min 95% B →42 min 95% B →42.1 min 30% B →45 min 30% B; flow rate: 50 ml/min; column temperature: RT; UV detection: 210 nm

### Method 10 (Preparative HPLC):

Instrument: Abimed Gilson Pump 305/306, Manometric Module 806; column: Grom-Sil 1200DS-4HE 10 μm, 250 mm×40 mm; eluent: A=water, B=acetonitrile; gradient 0.0 min 10% B →3 min 10% B →27 min 98% B →34 min 98% B →34.01 min 10% B→38 min 10% B; flow rate: 50 ml/min; column temperature: RT; UV detection: 214 nm

Method 11 (preparative HPLC):

Instrument: Abimed Gilson Pump 305/306, Manometric Module 806; column: Grom-Sil 120 ODS-4HE 10 μm, 250 mm×40 mm; eluent: A=water+0.75 ml formic acid/1 water, B=acetonitrile; gradient: 0.0 min 10% B →3 min 10% B →27 min 98% B →34 min 98% B →34.01 min 10% B →38 min 10% B; flow rate: 50 ml/min; column temperature: RT; UV detection: 214 nm

### Method 12 (LC-MS):

MS instrument type: Waters ZQ; HPLC instrument type: Waters Alliance 2795; column: Phenomenex Onyx Monolithic C18, 100 mm×3 mm; eluent A: 1 l water+0.5 ml 50% formic acid, eluent B: 1 l acetonitrile+0.5 ml 50% formic acid; gradient: 0.0 min 90% A →2 min 65% A →4.5 min 5% A →6 min 5% A; flow rate: 2 ml/min; oven: 40° C.; UV detection: 210 nm

### Starting Compounds and Intermediates

### Example 1A

### Methyl 6-(3-fluoro-4-methylphenyl)-4-oxo-1,4-dihydropyridine-3-carboxylate

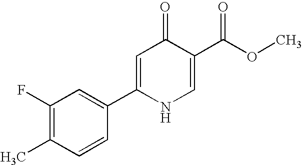

Under an argon atmosphere, 2.00 g (11.68 mmol) of methyl 2-[N,N-(dimethylamino)methylene]-3-oxobutanoate and 2.40 g (13.90 mmol) of 3-fluoro-4-methylbenzoyl chloride, each dissolved in 10 ml of THF, are simultaneously added dropwise at −70° C. with stirring to 28.04 ml (28.04 mmol) of a 1M solution of lithium hexamethyldisilazide in THF. The cooling bath is removed, and the mixture is left to stir for a further 5 min and admixed with 50 ml of diethyl ether. After adding 24 ml of acetic acid and 1.2 g of ammonium chloride, diethyl ether and THF are removed under reduced pressure on a rotary evaporator and the residue is heated to 60-65° C. for 1.5 h. For workup, the reaction mixture is partitioned between dichloromethane and water, and the organic phase is washed three times more with water and dried over magnesium sulfate. Concentration and drying of the residue under reduced pressure afford 3.86 g (approx. 57% pure by HPLC, corresponding to approx. 72% of theory) of the target compound, which is reacted without further purification.

LC-MS (method 2): Rₜ=1.81 min; m/z=262 [M+H]⁺.

### Example 2A

### Methyl 6-(2,3-difluorophenyl)-4-oxo-1,4-dihydropyridine-3-carboxylate

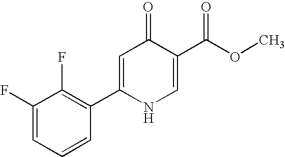

The title compound is prepared analogously to example 1A. The resulting crude product is purified by chromatography on about 200 g of silica gel first with cyclohexane, then with cyclohexane/ethyl acetate mixtures with an ethyl acetate content rising up to 33.3% as eluent. Starting from 1.64 g (9.58 mmol) of 2,3-difluorobenzoyl chloride, this affords 0.75 g (29% of theory) of the target compound.

¹H NMR (400 MHz, CDCl₃): δ=4.03 (s, 3H), 7.16-7.30 (m, 2H), 7.26 (s, 1H), 7.43 (d, 1H), 7.78 (ddt, 1H), 9.05 (s, 1H).

LC-MS (method 2): Rₜ=1.60 min; m/z=266 [M+H]⁺.

### Example 3A

### Methyl 4-chloro-6-(3-fluoro-4-methylphenyl)nicotinate

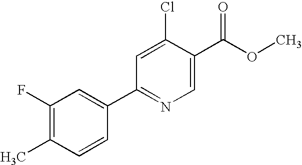

46.1 g (300 mmol) of phosphorus oxychloride are added to 3.86 g (14.78 mmol) of methyl 6-(3-fluoro-4-methylphenyl)-4-oxo-1,4-dihydropyridine-3-carboxylate from example 1A, and the mixture is heated to reflux for 1 h. After cooling, the mixture is worked up by adding it to warm water, extracting three times with dichloromethane, and washing the combined organic phases with aqueous sodium hydrogencarbonate solution, drying them over magnesium sulfate and concentrating. The crude product is purified by chromatography on silica gel (eluent: cyclohexane/ethyl acetate 4:1 →2:1). This affords 1.10 g (27% of theory) of the target compound.

¹H NMR (400 MHz, CDCl₃): δ=2.35 (d, 3H), 3.99 (s, 3H), 7.31 (t, 1H), 7.70 (dd, 1H), 7.74 (dd, 1H), 7.78 (s, 1H), 9.10 (s, 1H).

LC-MS (method 1): Rₜ=2.79 min; m/z=280 [M+H]⁺.

### Example 4A

### Methyl 4-chloro-6-(2,3-difluorophenyl)nicotinate

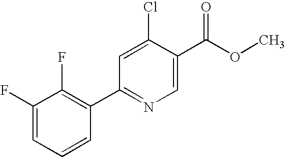

The title compound is prepared and worked up analogously to example 3A. Starting from 740 mg (2.79 mmol) of methyl 6-(2,3-difluorophenyl)-4-oxo-1,4-dihydropyridine-3-carboxylate from example 2A, this affords 665 mg (84% of theory) of the target compound. The product is used further without chromatographic purification.

¹H NMR (400 MHz, DMSO-d₆): δ=3.93 (s, 3H), 7.39 (tdd, 1H), 7.62 (dtd, 1H), 7.78 (ddt, 1H), 8.08 (d, 1H), 9.11 (s, 1H). LC-MS (method 1): Rₜ=2.61 min; m/z=284 [M+H]⁺.

### Example 5A

### Ethyl 4-chloro-6-(3,5-difluorophenyl)nicotinate

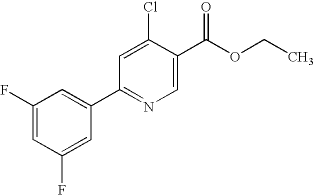

Under an argon atmosphere, 2.2 ml (1.1 mmol) of a 0.5M solution of 3,5-difluorophenylmagnesium bromide are added dropwise at 0° C. to 2.4 ml (1.2 mmol) of a 0.5M solution of zinc chloride in THF, and the mixture is left to stir at RT for a further 30 min A solution, prepared separately under argon, of 200 mg (0.91 mmol) of ethyl 2,4-dichloropyridine-5-carboxylate and 53 mg (0.045 mmol) of tetrakis(triphenylphosphine)palladium(0) in 2 ml of DMF is then added to the suspension formed. Subsequently, the mixture is left to stir at RT overnight. For workup, the mixture is stirred with 20 ml of water and 15 ml of ethyl acetate, and the mixture is filtered with suction through Celite. The organic phase is removed and concentrated, and the remaining residue is purified by preparative HPLC (method 10). This affords 114 mg (35% of theory) of the target compound.

¹H NMR (400 MHz, DMSO-d₆): δ=1.36 (t, 3H), 4.39 (q, 2H), 7.44 (tt, 1H), 7.90-8.00 (m, 2H), 8.42 (s, 1H), 9.05 (s, 1H).

LC-MS (method 5): Rₜ=4.03 min; m/z=298 [M+H]⁺.

### Example 6A

### Methyl 4-(2-chlorophenoxy)-6-(3-fluoro-4-methylphenyl)nicotinate

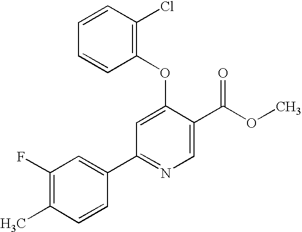

200 mg (0.72 mmol) of methyl 4-chloro-6-(3-fluoro-4-methylphenyl)nicotinate from example 3A are initially charged in 6.3 ml of DMF and admixed with 101 mg (0.76 mmol) of 2-chlorophenol and 296 mg (2.15 mmol) of potassium carbonate with stirring, and the mixture is stirred at 60° C. overnight. After the solid has been filtered off, the filtrate is purified by preparative HPLC (method 8). This affords 98 mg (37% of theory) of the target compound.

¹H NMR (400 MHz, CDCl₃): δ=2.30 (d, 3H), 3.95 (s, 3H), 6.86 (s, 1H), 7.17-7.30 (m, 3H), 7.37 (td, 1H), 7.47-7.57 (m, 3H), 9.13 (s, 1H).

LC-MS (method 2): Rₜ=2.88 min; m/z=372 [M+H]⁺.

### Example 7A

### Methyl 4-(2-chloro-4-methylphenoxy)-6-(3-fluoro-4-methylphenyl)nicotinate

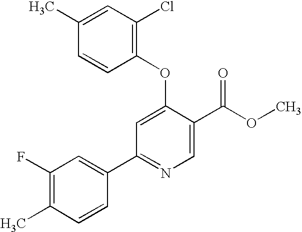

The title compound is prepared and purified analogously to example 6A. Starting from 200 mg (0.72 mmol) of methyl 4-chloro-6-(3-fluoro-4-methylphenyl)nicotinate from example 3A and 112 mg (0.79 mmol) of 2-chloro-4-methylphenol, this affords 97 mg (35% of theory) of the target compound.

¹H NMR (400 MHz, CDCl₃): δ=2.30 (d, 3H), 2.41 (s, 3H), 3.96 (s, 3H), 6.83 (s, 1H), 7.09 (d, 1H), 7.15 (dd, 1H), 7.21 (t, 1H), 7.35 (d, 1H), 7.48-7.53 (m, 2H), 9.11 (s, 1H).

LC-MS (method 2): Rₜ=3.03 min; m/z=386 [M+H]⁺.

### Example 8A

### Methyl 4-(2-chloro-5-methylphenoxy)-6-(3-fluoro-4-methylphenyl)nicotinate

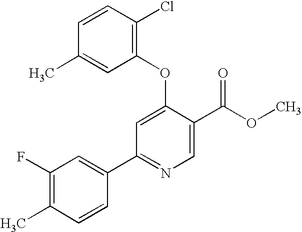

The title compound is prepared analogously to example 6A. Starting from 200 mg (0.72 mmol) of methyl 4-chloro-6-(3-fluoro-4-methylphenyl)nicotinate from example 3A and 112 mg (0.79 mmol) of 2-chloro-5-methylphenol, at a double purification by preparative HPLC (first by method 8, then by method 9), 23 mg (8% of theory) of the target compound are obtained.

¹H NMR (400 MHz, DMSO-d₆): δ=2.27 (d, 3H), 2.31 (s, 3H), 3.83 (s, 3H), 7.11-7.20 (m, 2H), 7.23 (s, 1H), 7.39 (t, 1H), 7.54 (d, 1H), 7.69 (dd, 1H), 7.77 (dd, 1H), 9.03 (s, 1H).

LC-MS (method 1): Rₜ=3.20 min; m/z=386 [M+H]⁺.

### Example 9A

### Methyl 4-(2-chloro-5-methoxyphenoxy)-6-(3-fluoro-4-methylphenyl)nicotinate

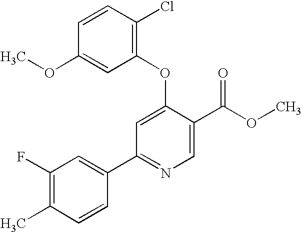

The title compound is prepared and purified analogously to example 6A. Starting from 150 mg (0.53 mmol) of methyl 4-chloro-6-(3-fluoro-4-methylphenyl)nicotinate from example 3A and 94 mg (0.59 mmol) of 2-chloro-5-methoxyphenol, this affords 89 mg (41% of theory) of the target compound.

¹H NMR (400 MHz, DMSO-d₆): δ=2.27 (d, 3H), 3.77 (s, 3H), 3.85 (s, 3H), 6.92-6.98 (m, 2H), 7.21 (s, 1H), 7.39 (t, 1H), 7.53-7.60 (m, 1H), 7.69 (dd, 1H), 7.77 (dd, 1H), 9.03 (s, 1H).

LC-MS (method 3): Rₜ=4.33 min; m/z=402 [M+H]⁺.

### Example 10A

### Methyl 4-(2-chloro-5-methoxyphenoxy)-6-(2,3-difluorophenyl)nicotinate

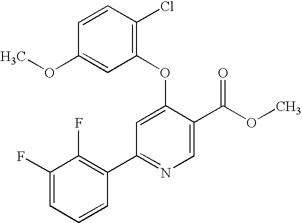

The title compound is prepared and purified analogously to example 6A. Starting from 150 mg (0.53 mmol) of methyl 4-chloro-6-(2,3-difluorophenyl)nicotinate from example 4A and 92 mg (0.58 mmol) of 2-chloro-5-methoxyphenol, this affords 100 mg (47% of theory) of the target compound.

¹H NMR (400 MHz, DMSO-d₆): δ=3.78 (s, 3H), 3.90 (s, 3H), 6.99 (dd, 1H), 7.04 (s, 1H), 7.06 (d, 1H), 7.30-7.38 (m, 1H), 7.50-7.59 (m, 1H), 7.59 (d, 1H), 7.74 (ddt, 1H), 9.08 (s, 1H).

LC-MS (method 3): Rₜ=4.12 min; m/z=406 [M+H]⁺.

### Example 11A

### Ethyl 4-(2-chlorophenoxy)-6-(3,5-difluorophenyl)nicotinate

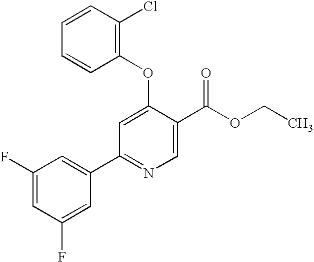

24 mg (0.19 mmol) of 2-chlorophenol and 70 mg (0.50 mmol) of potassium carbonate are added to a solution of 50 mg (0.17 mmol) of ethyl 4-chloro-6-(3,5-difluorophenyl)nicotinate from example 5A in 2.0 ml of DMF, and the mixture is stirred at 60° C. overnight. For workup and purification, the solid is filtered off and the filtrate is separated by preparative HPLC (method 10). This affords 61 mg (93% of theory) of the target compound.

¹H NMR (400 MHz, DMSO-d₆): δ=1.23 (t, 3H), 4.26 (q, 2H), 7.21 (dd, 1H), 7.31 (td, 1H), 7.35-7.44 (m, 2H), 7.55 (s, 1H), 7.66 (dd, 1H), 7.74-7.82 (m, 2H), 9.06 (s, 1H).

LC-MS (method 1): Rₜ=3.12 min; m/z=390 [M+H]⁺.

### Example 12A

### Ethyl 4-(2-chloro-5-methoxyphenoxy)-6-(3,5-difluorophenyl)nicotinate

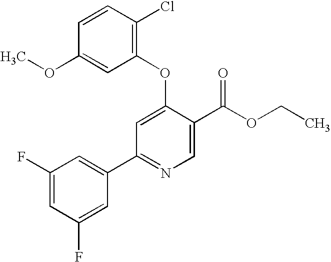

The title compound is prepared and purified analogously to example 11A. Starting from 50 mg (0.17 mmol) of ethyl 4-chloro-6-(3,5-difluorophenyl)nicotinate from example 5A and 29 mg (0.19 mmol) of 5-methoxy-2-chlorophenol, this affords 64 mg (91% of theory) of the target compound.

¹H NMR (400 MHz, DMSO-d₆): δ=1.23 (t, 3H), 3.75 (s, 3H), 4.29 (q, 2H), 6.85 (d, 1H), 6.91 (dd, 1H), 7.39 (tt, 1H), 7.48 (s, 1H), 7.54 (d, 1H), 7.73-7.82 (m, 2H), 9.04 (s, 1H).

LC-MS (method 3): Rₜ=4.47 min; m/z=420 [M+H]⁺.

### Example 13A

### Ethyl 6-chloro-4-(2-chlorophenoxy)nicotinate

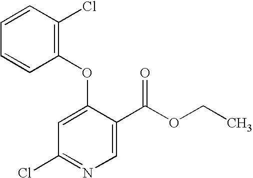

584 mg (4.54 mmol) of 2-chlorophenol and 1.88 g (13.63 mmol) of potassium carbonate are added to a solution of 1.00 g (4.54 mmol) of ethyl 4,6-dichloronicotinate in 15 ml of DMF, and the mixture is stirred at RT for about 70 h. For workup, the mixture is added to 250 ml of water and extracted four times with 50 ml of ethyl acetate each time, and the combined organic phases are washed once with saturated aqueous sodium chloride solution, dried over magnesium sulfate, filtered and concentrated under reduced pressure. The residue taken up in acetonitrile is purified by preparative HPLC (method 10). This affords 907 mg (64% of theory) of the target compound.

¹H NMR (400 MHz, DMSO-d₆): δ=1.28 (t, 3H), 4.31 (q, 2H), 6.76 (s, 1H), 7.37-7.43 (m, 2H), 7.46-7.52 (m, 1H), 7.67-7.72 (m, 1H), 8.80 (s, 1H).

LC-MS (method 1): Rₜ=2.73 min; m/z=312 [M+H]⁺.

### Example 14A

### Ethyl 4-(2-chlorophenoxy)-6-(4-methylphenyl)nicotinate

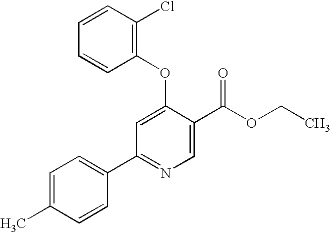

First 52.3 mg (0.38 mmol) of 4-methylphenylboronic acid, then 961 μl (1.92 mmol) of a 2M solution of potassium carbonate in water, after 10 minutes 45.0 mg (0.064 mmol) of bis(triphenylphosphine)palladium (II) chloride and 19.5 mg (0.064 mmol) of tri-2-tolylphosphine are added with stirring to a solution of 100 mg (0.32 mmol) of ethyl 6-chloro-4-(2-chlorophenoxy)-nicotinate (example 13A) in 2.00 ml of dioxane, and the mixture is then stirred at 60° C. overnight. For workup and purification, the reaction mixture is separated directly by preparative HPLC (method 10) to obtain 113 mg (96% of theory) of the target compound.

¹H NMR (400 MHz, DMSO-d₆): δ=1.24 (t, 3H), 2.34 (s, 3H), 4.28 (q, 2H), 7.20-7.31 (m, 4H), 7.33 (td, 1H), 7.44 (td, 1H), 7.68 (dd, 1H), 7.86, 7.88 (BB′ part of an AA′BB′system, 2H), 9.03 (s, 1H).

LC-MS (method 1): Rₜ=2.78 min; m/z=368 [M+H]⁺.

### Example 15A

### Methyl 4-(2-chloro-4-methoxyphenoxy)-6-(3-fluoro-4-methylphenyl)nicotinate

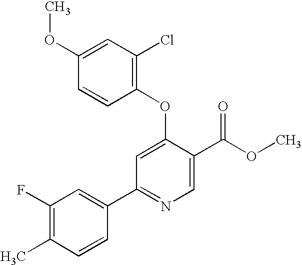

The title compound is prepared and purified analogously to example 6A. The purification is effected by preparative HPLC by method 9. Starting from 100 mg (0.36 mmol) of methyl 4-chloro-6-(3-fluoro-4-methylphenyl)nicotinate (example 3A) and 62 mg (0.39 mmol) of 2-chloro-4-methoxyphenol, this affords 92 mg (64% of theory) of the target compound.

¹H NMR (400 MHz, DMSO-d₆): δ=2.27 (d, 3H), 3.83 (s, 3H), 3.87 (s, 3H), 7.04 (dd, 1H), 7.08 (s, 1H), 7.27 (d, 1H), 7.35 (d, 1H), 7.38 (t, 1H), 7.64 (dd, 1H), 7.73 (dd, 1H), 9.00 (s, 1H).

LC-MS (method 1): Rₜ=3.08 min; m/z=402 [M+H]⁺.

### Example 16A

### Methyl 4-(2-chloro-4-trifluoromethoxyphenoxy)-6-(3-fluoro-4-methylphenyl)nicotinate

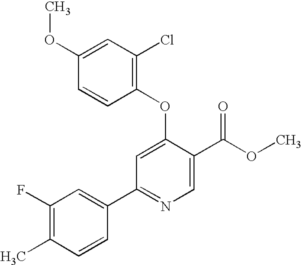

The title compound is prepared and purified analogously to example 15A. Starting from 90 mg (0.32 mmol) of methyl 4-chloro-6-(3-fluoro-4-methylphenyl)nicotinate (example 3A) and 75 mg (0.35 mmol) of 2-chloro-4-(trifluoromethoxy)phenol, this affords 48 mg (33% of theory) of the target compound.

¹H NMR (400 MHz, DMSO-d₆): δ=2.28 (s, 3H), 3.81 (s, 3H), 7.36 (d, 1H), 7.40 (t, 1H), 7.43 (dd, 1H), 7.52 (s, 1H), 7.80-7.89 (m, 3H), 9.06 (s, 1H).

LC-MS (method 1): Rₜ=3.28 min; m/z=456 [M+H]⁺.

### Example 17A

### Ethyl 4-chloro-6-(3-fluorophenyl)nicotinate

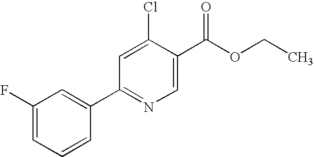

The title compound is prepared, worked up and purified analogously to example 5A, except that the reaction time is about 40 h. Starting from 200 mg (0.91 mmol) of ethyl 4,6-dichloronicotinate and 1.09 ml (1.09 mmol) of a 1M solution of 3-fluorophenylmagnesium bromide in THF, this affords 143 mg (47% of theory) of the target compound.

¹H NMR (400 MHz, DMSO-d₆): δ=1.36 (t, 3H), 4.38 (q, 2H), 7.38 (td, 1H), 7.59 (td, 1H), 8.03 (ddd, 1H), 8.07 (d, 1H), 8.35 (s, 1H), 9.05 (s, 1H).

LC-MS (method 12): Rₜ=3.88 min; m/z=280 [M+H]⁺.

### Example 18A

### Ethyl 4-(2-chlorophenoxy)-6-(3-fluorophenyl)nicotinate

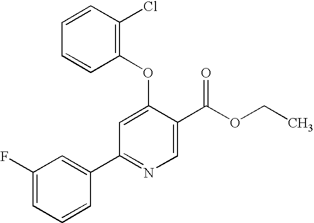

62 mg (0.22 mmol) of ethyl 4-chloro-6-(3-fluorophenyl)nicotinate (example 17A) are initially charged in 3.0 ml of DMF and admixed with 31 mg (0.24 mmol) of 2-chlorophenol and 92 mg (0.67 mmol) of potassium carbonate with stirring, and the mixture is stirred first at 60° C. for 9 h, then at 80° C. for another 4 h. After filtration from the solid, the filtrate is purified directly by preparative HPLC (method 10). This affords 68 mg (82% of theory) of the target compound.

¹H NMR (400 MHz, DMSO-d₆): δ=1.24 (t, 3H), 4.28 (q, 2H), 7.26 (td, 1H), 7.29-7.37 (m, 2H), 7.39 (s, 1H), 7.43 (td, 1H), 7.53 (td, 1H), 7.67 (dd, 1H), 7.79-7.88 (m, 2H), 9.06 (s, 1H).

LC-MS (method 1): Rₜ=3.09 min; m/z=372 [M+H]⁺.

### Example 19A

### Ethyl 6-(3-fluorophenyl)-2-methyl-4-oxo-1,4-dihydropyridine-3-carboxylate

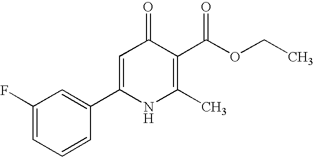

338 mg (2.62 mmol) of ethyl3-aminocrotonate and 1.00 g of molecular sieve (5 Å) are added to a solution of 500 mg (2.40 mmol) of ethyl 3-(3-fluorophenyl)-3-oxopropionate in 2.8 ml of xylene, and the mixture is stirred under reflux overnight. For workup, the molecular sieve is filtered off and is washed with 25 ml of a 1:1 mixture of chloroform and methanol. The combined organic solutions are concentrated and the residue is purified by preparative HPLC (method A). This affords 151 mg (23% of theory) of the target compound.

LC-MS (method 3): Rₜ=2.14 min; m/z=276 [M+H]⁺.

### Example 20A

### Ethyl 4-chloro-6-(3-fluorophenyl)-2-methylnicotinate

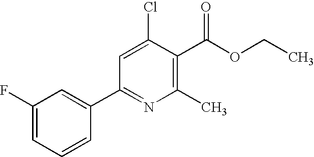

The title compound is prepared and worked up analogously to example 4A. Starting from 150 mg (0.55 mmol) of ethyl 6-(3-fluorophenyl)-2-methyl-4-oxo-1,4-dihydropyridine-3-carboxylate (example 19A), this affords 140 mg (87% of theory) of the target compound.

¹H NMR (400 MHz, DMSO-d₆): δ=1.35 (t, 3H), 2.56 (s, 3H), 4.43 (q, 2H), 7.35 (td, 1H), 7.57 (td, 1H), 7.97 (ddd, 1H), 8.02 (d, 1H), 8.18 (s, 1H).

LC-MS (method 3): Rₜ=4.26 min; m/z=294 [M+H]⁺.

### Example 21A

### Ethyl 4-(2-chlorophenoxy)-6-(3-fluorophenyl)-2-methylnicotinate

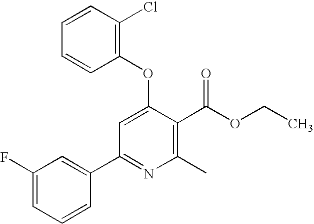

260 mg (0.89 mmol) of ethyl 4-chloro-6-(3-fluorophenyl)-2-methylnicotinate (example 20A) are initially charged in 11.1 ml of DMF and admixed with 137 mg (1.06 mmol) of 2-chlorophenol and 367 mg (2.66 mmol) of potassium carbonate with stirring, and the mixture is stirred at 100° C. overnight. Another 100 mg (0.72 mmol) of potassium carbonate are added and the mixture is heated to 100° C. for a further 20 h. After filtration from the solid, the filtrate is purified by double preparative HPLC (method 8 each time). This affords 167 mg (49% of theory) of the target compound.

¹H NMR (400 MHz, DMSO-d₆): δ=1.26 (t, 3H), 2.58 (s, 3H), 4.32 (q, 2H), 7.16 (s, 1H), 7.26-7.37 (m, 3H), 7.44 (td, 1H), 7.50 (td, 1H), 7.66 (dd, 1H), 7.73-7.82 (m, 2H).

LC-MS (method 1): Rₜ=3.29 min; m/z=386 [M+H]⁺.

### Example 22A

### Ethyl 4-chloro-6-(4-fluorophenyl)nicotinate

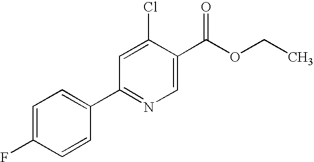

The title compound is prepared, worked up and purified analogously to example 17A. Starting from 200 mg (0.91 mmol) of ethyl 4,6-dichloronicotinate and 1.09 ml (1.09 mmol) of a 1M solution of 4-fluorophenylmagnesium bromide in THF, this affords 140 mg (46% of theory) of the target compound.

¹H NMR (400 MHz, DMSO-d₆): δ=1.36 (t, 3H), 4.38 (q, 2H), 7.34-7.41 (m, 2H), 8.24-8.30 (m, 3H), 9.03 (s, 1H).

LC-MS (method 12): Rₜ=3.86 min; m/z=280 [M+H]⁺.

### Example 23A

### Ethyl 4-(2-chlorophenoxy)-6-(4-fluorophenyl)nicotinate

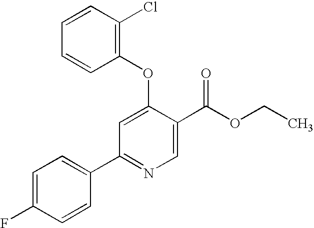

The title compound is prepared, worked up and purified analogously to example 11A, except that the reaction time is 15 h. Starting from 66 mg (0.24 mmol) of ethyl 4-chloro-6-(4-fluorophenyl)-nicotinate (example 22A) and 33 mg (0.26 mmol) of 2-chlorophenol, this affords 80 mg (91% of theory) of the target compound.

¹H NMR (400 MHz, DMSO-d₆): δ=1.24 (t, 3H), 4.28 (q, 2H), 7.25-7.36 (m, 5H), 7.44 (td, 1H), 7.68 (dd, 1H), 8.01-8.08 (m, 2H), 9.04 (s, 1H).

LC-MS (method 1): Rₜ=3.06 min; m/z=372 [M+H]⁺.

### Example 24A

### Methyl 4-(2-chloro-5-methylphenoxy)-6-(2,3-difluorophenyl)nicotinate

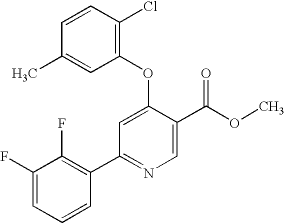

The title compound is prepared and worked up analogously to example 11A. For the purification, the crude product is separated by preparative HPLC (method 9). Starting from 125 mg (0.44 mmol) of 4-chloro-6-(2,3-difluorophenyl)nicotinate (example 4A) and 69 mg (0.49 mmol) of 2-chloro-5-methylphenol, this affords 69 mg (40% of theory) of the target compound.

¹H NMR (400 MHz, DMSO-d₆): δ=2.33 (s, 3H), 3.89 (s, 3H), 7.02 (s, 1H), 7.22 (dd, 1H), 7.26 (d, 1H), 7.34 (tdd, 1H), 7.50-7.57 (m, 1H), 7.57 (d, 1H), 7.75 (ddt, 1H), 9.08 (s, 1H).

LC-MS (method 12): Rₜ=4.08 min; m/z=390 [M+H]⁺.

### Example 25A

### Methyl 4-(2-chloro-4-methoxyphenoxy)-6-(2,3-difluorophenyl)nicotinate

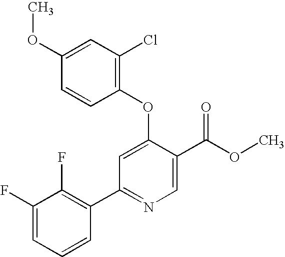

The title compound is prepared, worked up and purified analogously to example 24A. Starting from 125 mg (0.44 mmol) of methyl 4-chloro-6-(2,3-difluorophenyl)nicotinate (example 4A) and 77 mg (0.49 mmol) of 2-chloro-4-methoxyphenol, this affords 124 mg (69% of theory) of the target compound.

¹H NMR (400 MHz, DMSO-d₆): δ=3.83 (s, 3H), 3.91 (s, 3H), 6.97 (s, 1H), 7.06 (dd, 1H), 7.29 (d, 1H), 7.33 (tdd, 1H), 7.40 (d, 1H), 7.54 (dtd, 1H), 7.73 (ddt, 1H), 9.06 (s, 1H).

LC-MS (method 1): Rₜ=2.90 min; m/z=406 [M+H]⁺.

### Example 26A

### Methyl 4-(2-chlorophenoxy)-6-(2,3-difluorophenyl)nicotinate

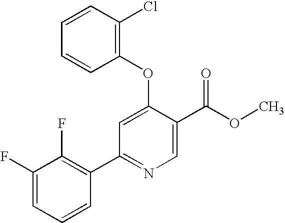

The title compound is prepared, worked up and purified analogously to example 24A. Starting from 125 mg (0.44 mmol) of methyl 4-chloro-6-(2,3-difluorophenyl)nicotinate (example 4A) and 62 mg (0.49 mmol) of 2-chlorophenol, this affords 75 mg (45% of theory) of the target compound.

¹H NMR (400 MHz, DMSO-d₆): δ=3.90 (s, 3H), 7.03 (s, 1H), 7.06 (dd, 1H), 7.33 (tdd, 1H), 7.38-7.45 (m, 2H), 7.47-7.59 (m, 2H), 7.71 (dd, 1H), 7.75 (ddt, 1H), 9.09 (s, 1H).

LC-MS (method 1): Rₜ=2.89 min; m/z=376 [M+H]⁺.

### Example 27A

### Methyl 6-(2,4-difluorophenyl)-4-oxo-1,4-dihydropyridine-3-carboxylate

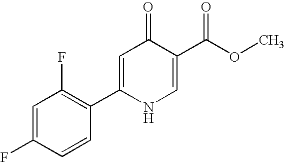

The title compound is prepared and worked up analogously to example 1A. For purification, the resulting crude product is separated by chromatography on 200 g of silica gel (eluent gradient: cyclohexane →4:1 cyclohexane/ethyl acetate). Starting from 1.50 g (8.76 mmol) of methyl 2-[N,N-(dimethylamino)methylene]-3-oxobutanoate and 1.84 g (10.4 mmol) of 2,4-difluorobenzoyl chloride, this affords 260 mg (11% of theory) of the target compound.

LC-MS (method 1): Rₜ=1.72 min; m/z=266 [M+H]⁺.

### Example 28A

### Methyl 4-chloro-6-(2,4-difluorophenyl)nicotinate

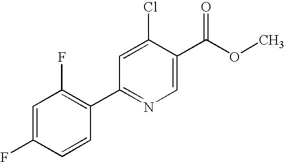

The title compound is prepared and worked up analogously to example 3A. Starting from 260 mg (2.79 mmol) of methyl 6-(2,4-difluorophenyl)-4-oxo-1,4-dihydropyridine-3-carboxylate (example 27A), this affords 240 mg (86% of theory) of the target compound. The product is used further without chromatographic purification.

¹H NMR (400 MHz, DMSO-d₆): δ=3.93 (s, 3H), 7.29 (td, 1H), 7.48 (ddd, 1H), 8.02 (s, 1H), 8.07 (td, 1H), 9.09 (s, 1H).

LC-MS (method 1): Rₜ=2.71 min; m/z=284 [M+H]⁺.

### Example 29A

### Methyl 4-(2-chlorophenoxy)-6-(2,4-difluorophenyl)nicotinate

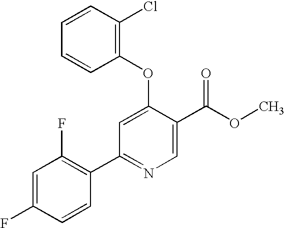

The title compound is prepared, worked up and purified analogously to example 24A. Starting from 120 mg (0.42 mmol) of methyl 4-chloro-6-(2,4-difluorophenyl)nicotinate (example 28A) and 59 mg (0.47 mmol) of 2-chlorophenol, this affords 110 mg (69% of theory) of the target compound.

¹H NMR (400 MHz, DMSO-d₆): δ=3.89 (s, 3H), 6.97 (s, 1H), 7.24 (td, 1H), 7.34 (ddd, 1H), 7.39-7.45 (m, 2H), 7.50 (ddd, 1H), 7.71 (dd, 1H), 8.07 (td, 1H), 9.07 (s, 1H).

LC-MS (method 1): Rₜ=2.94 min; m/z=376 [M+H]⁺.

### Example 30A

### Ethyl 4-chloro-6-(3-chlorophenyl)nicotinate

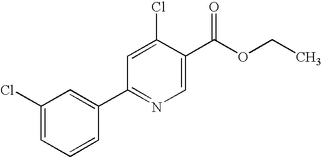

Under an argon atmosphere, 2.18 ml (1.09 mmol) of a 0.5M solution of 3-chlorophenylzinc iodide in THF and 53 mg (0.045 mmol) of tetrakis(triphenylphosphine)palladium(0) are added at RT to a solution of 200 mg (0.91 mmol) of ethyl 4,6-dichloronicotinate. Subsequently, the mixture is left to continue to stir at RT overnight. For workup, the mixture is stirred with 40 ml of water and 20 ml of ethyl acetate and filtered with suction through 2 g of Celite. The organic phase is removed and concentrated, and the remaining residue is purified by preparative HPLC (method 10). The combined product fractions are purified further by flash chromatography on silica gel in 50:1 cyclohexane/ethyl acetate. This affords 132 mg (49% of theory) of the target compound.

¹H NMR (400 MHz, DMSO-d₆): δ=1.36 (t, 3H), 4.39 (q, 2H), 7.54-7.63 (m, 2H), 8.18 (dt, 1H), 8.24-8.27 (m, 1H), 8.37 (s, 1H), 9.05 (s, 1H).

LC-MS (method 3): Rₜ=4.35 min; m/z=296 [M+H]⁺.

### Example 31A

### Ethyl 4-(2-chlorophenoxy)-6-(3-chlorophenyl)nicotinate

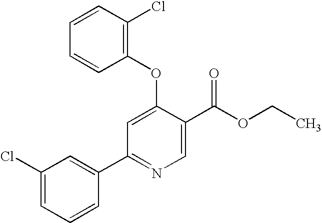

64 mg (0.22 mmol) of ethyl 4-chloro-6-(3-chlorophenyl)nicotinate (example 30A) are initially charged in 3.0 ml of DMF and admixed with 31 mg (0.24 mmol) of 2-chlorophenol and 90 mg (0.65 mmol) of potassium carbonate with stirring. The mixture is stirred first at 60° C. overnight, then at 100° C. for a further 2 h to complete the conversion. After the solid has been filtered off, the filtrate is purified directly by preparative HPLC (method 10). This affords 70 mg (83% of theory) of the target compound.

¹H NMR (400 MHz, DMSO-d₆): δ=1.24 (t, 3H), 4.28 (q, 2H), 7.25 (dd, 1H), 7.32 (td, 1H), 7.41 (s, 1H), 7.43 (td, 1H), 7.48-7.58 (m, 2H), 7.67 (dd, 1H), 7.93 (dt, 1H), 8.06-8.10 (m, 1H), 9.06 (s, 1H).

LC-MS (method 12): Rₜ=4.36 min; m/z=388 [M+H]⁺.

### Example 32A

### Ethyl 4-(2-chloro-4-trifluoromethoxyphenoxy)-6-(3-chlorophenyl)nicotinate

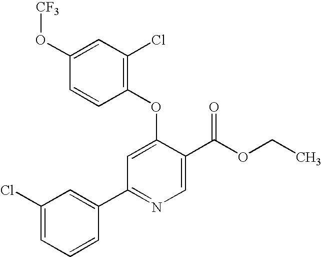

64 mg (0.22 mmol) of ethyl 4-chloro-6-(3-chlorophenyl)nicotinate (example 30A) are initially charged in 3.0 ml of DMF and admixed with 50 mg (0.24 mmol) of 2-chloro-4-(trifluoromethoxy)phenol and 90 mg (0.65 mmol) of potassium carbonate with stirring. The mixture is stirred first at 60° C. overnight, then at 100° C. for a further 3 h to complete the conversion. After the solid has been filtered off, the filtrate is purified directly by preparative HPLC (method 10). This affords 90 mg (88% of theory) of the target compound.

¹H NMR (400 MHz, DMSO-d₆): δ=1.20 (t, 3H), 4.26 (q, 2H), 7.30 (d, 1H), 7.42 (dd, 1H), 7.49-7.59 (m, 2H), 7.69 (s, 1H), 7.83 (d, 1H), 8.05 (br. d, 1H), 8.16 (br. s, 1H), 9.09 (s, 1H).

LC-MS (method 12): Rₜ=4.61 min; m/z=472 [M+H]⁺.

### Example 33A

### Ethyl 6-(3-chloro-4-fluorophenyl)-4-(2-chlorophenoxy)nicotinate

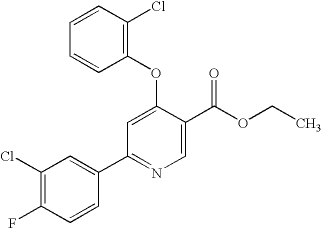

The title compound is prepared, worked up and purified analogously to example 14A. Starting from 100 mg (0.32 mmol) of ethyl 6-chloro-4-(2-chlorophenoxy)nicotinate (example 13A) and 67 mg (0.38 mmol) of 3-chloro-4-fluorophenylboronic acid, this affords 111 mg (85% of theory) of the target compound.

¹H NMR (400 MHz, DMSO-d₆): δ=1.23 (t, 3H), 4.27 (q, 2H), 7.23 (dd, 1H), 7.31 (td, 1H), 7.42 (td, 1H), 7.47 (s, 1H), 7.53 (t, 1H), 7.67 (dd, 1H), 8.03 (ddd, 1H), 8.27 (dd, 1H), 9.05 (s, 1H).

LC-MS (method 1): Rₜ=2.91 min; m/z=406 [M+H]⁺.

### Example 34A

### Ethyl 6-(4-bromo-2-fluorophenyl)-4-chloronicotinate

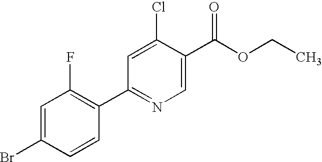

The title compound is prepared, worked up and purified analogously to example 30A. Starting from 200 mg (0.91 mmol) of ethyl 4,6-dichloronicotinate and 2.18 ml (1.09 mmol) of a 0.5M solution of 4-bromo-2-fluorophenylzinc iodide in THF, this affords 107 mg (33% of theory) of the target compound.

¹H NMR (400 MHz, DMSO-d₆): δ=1.36 (t, 3H), 4.39 (q, 2H), 7.62 (dd, 1H), 7.78 (dd, 1H), 7.95 (t, 1H), 8.03 (d, 1H), 9.09 (s, 1H).

LC-MS (method 1): Rₜ=3.16 min; m/z=358 [M+H]⁺.

### Example 35A

### Ethyl 6-(4-bromo-2-fluorophenyl)-4-(2-chlorophenoxy)nicotinate

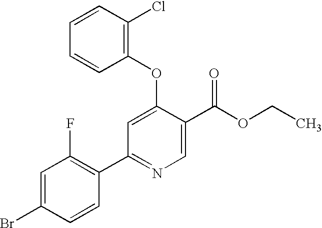

The title compound is prepared, worked up and purified analogously to example 11A. Starting from 50 mg (0.14 mmol) of ethyl 6-(4-bromo-2-fluorophenyl)-4-chloronicotinate (example 34A) and 20 mg (0.15 mmol) of 2-chlorophenol, this affords 53 mg (84% of theory) of the target compound.

¹H NMR (400 MHz, DMSO-d₆): δ=1.30 (t, 3H), 4.34 (q, 2H), 7.04 (s, 1H), 7.36-7.42 (m, 2H), 7.49 (ddd, 1H), 7.57 (dd, 1H), 7.66 (dd, 1H), 7.68-7.72 (m, 1H), 7.96 (t, 1H), 9.07 (s, 1H).

LC-MS (method 3): Rₜ=4.64 min; m/z=450 [M+H]⁺.

### Working Examples

### Example 1

### 4-(2-Chlorophenoxy)-6-(3-fluoro-4-methylphenyl)nicotinic acid

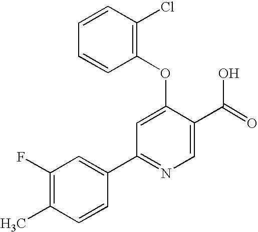

9.2 mg (0.38 mmol) of lithium hydroxide in 1.00 ml of water are added to 95 mg (0.26 mmol) of methyl 4-(2-chlorophenoxy)-6-(3-fluoro-4-methylphenyl)nicotinate from example 6A in 5 ml of THF, and the mixture is stirred at RT overnight. For workup and purification, the mixture is adjusted to pH 3 with 0.1N hydrochloric acid and partitioned between water and ethyl acetate, and the aqueous phase is extracted twice more with ethyl acetate. The combined organic phases are dried over magnesium sulfate and concentrated. Drying under reduced pressure affords 87 mg (95% of theory) of the target compound.

¹H NMR (400 MHz, DMSO-d₆): δ=2.27 (br. s, 3H), 7.22 (s, 1H), 7.28 (dd, 1H), 7.31-7.41 (m, 2H), 7.44 (td, 1H), 7.67 (dt, 2H), 7.76 (dd, 1H), 9.03 (s, 1H), 13.35 (br. s, 1H).

LC-MS (method 1): Rₜ=2.66 min; m/z=358 [M+H]⁺.

### Example 2

### 4-(2-Chloro-4-methylphenoxy)-6-(3-fluoro-4-methylphenyl)nicotinic acid

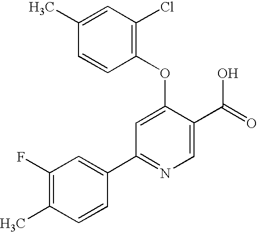

The title compound is prepared analogously to example 1. Starting from methyl 4-(2-chloro-4-methylphenoxy)-6-(3-fluoro-4-methylphenyl)nicotinate from example 7A, this affords 87 mg (95% of theory) of the target compound.

¹H NMR (400 MHz, DMSO-d₆): δ=2.26 (br. s, 3H), 2.36 (s, 3H), 7.12 (s, 1H), 7.21 (d, 1H), 7.26 (dd, 1H), 7.37 (t, 1H), 7.50 (d, 1H), 7.64 (dd, 1H), 7.73 (dd, 1H), 9.00 (s, 1H), 13.33 (br. s, 1H).

LC-MS (method 1): Rₜ=2.81 min; m/z=372 [M+H]⁺.

### Example 3

### 4-(2-Chloro-5-methylphenoxy)-6-(3-fluoro-4-methylphenyl)nicotinic acid

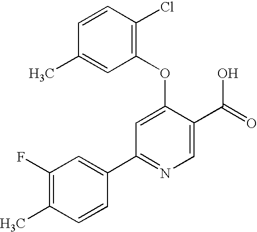

The title compound is prepared analogously to example 1. For purification, the solid initially obtained is stirred with 1.5 ml of diethyl ether, filtered off, washed again with diethyl ether and dried under reduced pressure. Starting from 22 mg (0.057 mmol) of methyl 4-(2-chloro-5-methylphenoxy)-6-(3-fluoro-4-methylphenyl)nicotinate from example 8A, this affords 9 mg (42% of theory) of the target compound.

¹H NMR (400 MHz, DMSO-d₆): δ=2.27 (s, 3H), 2.31 (s, 3H), 7.08-7.22 (m, 3H), 7.38 (t, 1H), 7.53 (d, 1H), 7.67 (d, 1H), 7.75 (d, 1H), 9.01 (s, 1H), 13.29 (br. s, 1H).

LC-MS (method 3): Rₜ=3.91 min; m/z=372 [M+H]⁺.

### Example 4

### 4-(2-Chloro-5-methoxyphenoxy)-6-(3-fluoro-4-methylphenyl)nicotinic acid

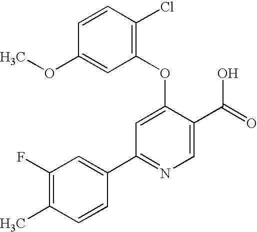

The title compound is prepared analogously to example 1. Starting from 82 mg (0.20 mmol) of methyl 4-(2-chloro-5-methoxyphenoxy)-6-(3-fluoro-4-methylphenyl)nicotinate from example 9A, this affords 27 mg (34% of theory) of the target compound.

¹H NMR (400 MHz, DMSO-d₆): δ=2.27 (s, 3H), 3.77 (s, 3H), 6.89-6.97 (m, 2H), 7.17 (s, 1H), 7.38 (t, 1H), 7.55 (d, 1H), 7.67 (dd, 1H), 7.76 (dd, 1H), 9.01 (s, 1H), 13.34 (br. s, 1H).

LC-MS (method 5): Rₜ=3.62 min; m/z=388 [M+H]⁺.

### Example 5

### 4-(2-Chloro-5-methoxyphenoxy)-6-(2,3-difluorophenyl)nicotinic acid

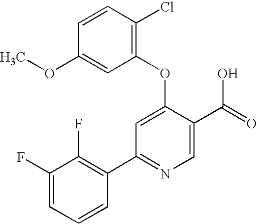

The title compound is prepared analogously to example 1. Starting from 95 mg (0.23 mmol) of methyl 4-(2-chloro-5-methoxyphenoxy)-6-(2,3-difluorophenyl)nicotinate from example 10A, this affords 88 mg (96% of theory) of the target compound.

¹H NMR (400 MHz, DMSO-d₆): δ=3.78 (s, 3H), 6.98 (dd, 1H), 7.00 (s, 1H), 7.04 (d, 1H), 7.29-7.37 (m, 1H), 7.49-7.59 (m, 1H), 7.58 (d, 1H), 7.74 (ddt, 1H), 9.06 (s, 1H), 13.48 (br. s, 1H).

LC-MS (method 1): Rₜ=2.53 min; m/z=392 [M+H]⁺.

### Example 6

### 4-(2-Chlorophenoxy)-6-(3,5-difluorophenyl)nicotinic acid

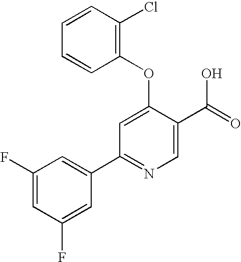

0.21 ml of a 1M aqueous solution of lithium hydroxide and 0.5 ml of water are added to 55 mg (0.141 mmol) of ethyl 4-(2-chlorophenoxy)-6-(3,5-difluorophenyl)nicotinate from example 11A, dissolved in 2 ml of THF, and the mixture is stirred at RT overnight. For workup and purification, the mixture is acidified with 1N hydrochloric acid and separated directly by preparative HPLC (method 11). This affords 44 mg (86% of theory) of the target compound.

¹H NMR (400 MHz, DMSO-d₆): δ=7.24 (dd, 1H), 7.32 (td, 1H), 7.38 (tt, 1H), 7.42 (s, 1H), 7.43 (td, 1H), 7.66 (dd, 1H), 7.70-7.78 (m, 2H), 9.05 (s, 1H), 13.45 (br. s, 1H).

LC-MS (method 3): Rₜ=3.69 min; m/z=362 [M+H]⁺.

### Example 7

### 4-(2-Chloro-5-methoxyphenoxy)-6-(3,5-difluorophenyl)nicotinic acid

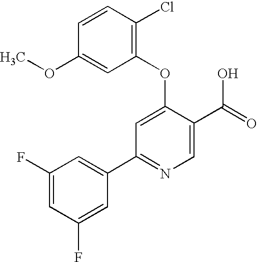

The title compound is prepared analogously to example 1. For purification, after acidification of the reaction mixture to pH 4-5 with 1N hydrochloric acid, the mixture is separated directly by preparative HPLC (method 11). Starting from 58 mg (0.14 mmol) of ethyl 4-(2-chloro-5-methoxyphenoxy)-6-(3,5-difluorophenyl)nicotinate from example 12A, this affords 48 mg (89% of theory) of the target compound.

¹H NMR (400 MHz, DMSO-d₆): δ=3.76 (s, 3H), 6.88 (d, 1H), 6.93 (dd, 1H), 7.35 (s, 1H), 7.38 (tt, 1H), 7.54 (d, 1H), 7.69-7.78 (m, 2H), 9.03 (s, 1H), 13.44 (br. s, 1H).

LC-MS (method 3): Rₜ=3.75 min; m/z=392 [M+H]⁺.

### Example 8

### 4-(2-Chlorophenoxy)-6-(4-methylphenyl)nicotinic acid

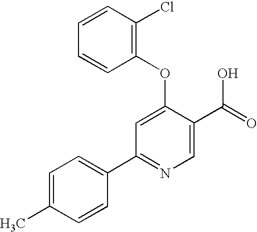

The title compound is prepared and purified analogously to example 6. Starting from 109 mg (0.30 mmol) of ethyl 4-(2-chlorophenoxy)-6-(4-methylphenyl)nicotinate (example 14A), this affords 88 mg (87% of theory) of the target compound.

¹H NMR (400 MHz, DMSO-d₆): 2.33 (s, 3H), 7.09 (s, 1H), 7.26, 7.28 (AA′ part of an AA′BB′ system, 2H), 7.31 (dd, 1H), 7.35 (td, 1H), 7.46 (td, 1H), 7.68 (dd, 1H), 7.81, 7.83 (BB′ part of an AA′BB′ system, 2H), 9.02 (s, 1H), 13.31 (br. S, 1H).

LC-MS (method 1): Rₜ=2.57 min; m/z=340 [M+H]⁺.

### Example 9

### 4-(2-Chloro-4-methoxyphenoxy)-6-(3-fluoro-4-methylphenyl)nicotinic acid

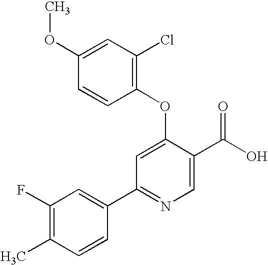

The title compound is prepared and worked up analogously to example 1. Starting from 90 mg (0.22 mol) of methyl 4-(2-chloro-4-methoxyphenoxy)-6-(3-fluoro-4-methylphenyl)nicotinate (example 15A), this affords 87 mg (100% of theory) of the target compound.

¹H NMR (400 MHz, DMSO-d₆): δ=2.26 (s, 3H), 3.83 (s, 3H), 7.03 (s, 1H), 7.04 (dd, 1H), 7.27 (d, 1H), 7.33 (d, 1H), 7.37 (t, 1H), 7.61 (dd, 1H), 7.72 (dd, 1H), 8.98 (s, 1H), 13.32 (br. s, 1H).

LC-MS (method 12): Rₜ=3.58 min; m/z=388 [M+H]⁺.

### Example 10

### 4-(2-Chloro-4-trifluoromethoxyphenoxy)-6-(3-fluoro-4-methylphenyl)nicotinic acid

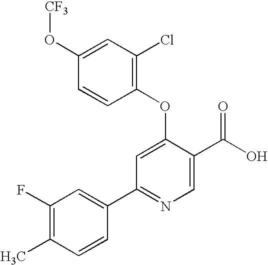

The title compound is prepared and worked up analogously to example 1. The resulting residue is also washed with cyclohexane for purification. Starting from 45 mg (0.10 mmol) of methyl 4-(2-chloro-4-trifluoromethoxyphenoxy)-6-(3-fluoro-4-methylphenyl)nicotinate (example 16A), this affords 33 mg (76% of theory) of the target compound.

¹H NMR (400 MHz, DMSO-d₆): δ=2.28 (s, 3H), 7.31 (d, 1H), 7.36-7.45 (m, 2H), 7.50 (s, 1H), 7.79-7.88 (m, 3H), 9.05 (s, 1H), 13.38 (br. s, 1H).

LC-MS (method 12): Rₜ=3.95 min; m/z=422 [M+H]⁺.

### Example 11

### 4-(2-Chlorophenoxy)-6-(3-fluorophenyl)nicotinic acid

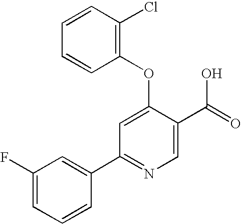

169 μl (0.254 mmol) of a 1.5 M solution of lithium hydroxide in water and also 1 ml of water are added to a solution of 63 mg (0.17 mmol) of ethyl 4-(2-chlorophenoxy)-6-(3-fluorophenyl)nicotinate (example 18A) in 4.0 ml of THF, and the mixture is stirred at RT overnight. For work up, the mixture is acidified to about pH 4-5 with 1N hydrochloric acid and separated by preparative HPLC for purification (method 11). This affords 52 mg (89% of theory) of the target compound.

¹H NMR (400 MHz, DMSO-d₆): δ=7.25 (s, 1H), 7.27-7.37 (m, 3H), 7.45 (t, 1H), 7.51 (q, 1H), 7.67 (d, 1H), 7.77 (d, 1H), 7.81 (br. d, 1H), 9.04 (s, 1H), 13.38 (br. s, 1H).

LC-MS (method 1): Rₜ=2.52 min; m/z=344 [M+H]⁺.

### Example 12

### 4-(2-Chlorophenoxy)-6-(3-fluorophenyl)-2-methylnicotinic acid

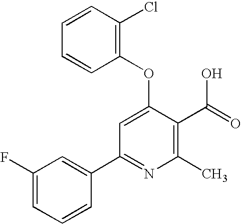

80 mg (0.21 mmol) of ethyl 4-(2-chlorophenoxy)-6-(3-fluorophenyl)-2-methylnicotinate (example 21A) and 116 mg (2.07 mmol) of potassium hydroxide in 8.0 ml of ethanol with a few drops of water are stirred under reflux overnight. For work up, after cooling, the mixture is added to water and acidified with 1N hydrochloric acid. After extracting three times with ethyl acetate, the combined organic phases are dried over magnesium sulfate, filtered and concentrated. This affords 70 mg (94% of theory) of the target compound.

¹H NMR (400 MHz, DMSO-d₆): δ=2.60 (s, 3H), 7.07 (s, 1H), 7.24-7.38 (m, 3H), 7.42-7.52 (m, 2H), 7.67 (dd, 1H), 7.73 (d, 1H), 7.73-7.80 (m, 1H), 13.64 (br. s, 1H).

LC-MS (method 1): Rₜ=2.32 min; m/z=358 [M+H]⁺.

### Example 13

### 4-(2-Chlorophenoxy)-6-(4-fluorophenyl)nicotinic acid

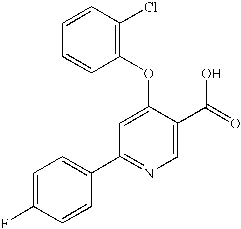

The title compound is prepared, worked up and purified analogously to example 11. Starting from 72 mg (0.19 mmol) of ethyl 4-(2-chlorophenoxy)-6-(4-fluorophenyl)nicotinate (example 23A), this affords 63 mg (95% of theory) of the target compound.

¹H NMR (400 MHz, DMSO-d₆): δ=7.15 (s, 1H), 7.26-7.34 (m, 3H), 7.35 (td, 1H), 7.46 (td, 1H), 7.68 (dd, 1H), 7.97-8.04 (m, 2H), 9.03 (s, 1H), 13.34 (br. s, 1H).

LC-MS (method 3): Rₜ=3.49 min; m/z=344 [M+H]⁺.

### Example 14

### 4-(2-Chloro-5-methylphenoxy)-6-(2,3-difluorophenyl)nicotinic acid

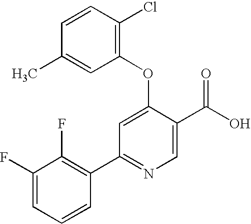

The title compound is prepared, worked up and purified analogously to example 10. Starting from 65 mg (0.17 mmol) of methyl 4-(2-chloro-5-methylphenoxy)-6-(2,3-difluorophenyl)nicotinate (example 24A), this affords 58 mg (93% of theory) of the target compound.

¹H NMR (400 MHz, DMSO-d₆): δ=2.33 (s, 3H), 6.98 (s, 1H), 7.21 (d, 1H), 7.24 (s, 1H), 7.33 (q, 1H), 7.49-7.60 (m, 2H), 7.74 (t, 1H), 9.06 (s, 1H), 13.48 (br. s, 1H).

LC-MS (method 12): Rₜ=3.52 min; m/z=376 [M+H]⁺.

### Example 15

### 4-(2-Chloro-4-methoxyphenoxy)-6-(2,3-difluorophenyl)nicotinic acid

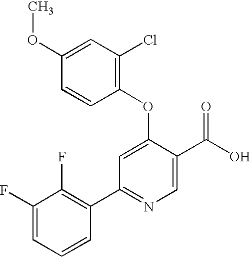

The title compound is prepared and worked up analogously to example 1. Starting from 120 mg (0.30 mmol) of methyl 4-(2-chloro-4-methoxyphenoxy)-6-(2,3-difluorophenyl)nicotinate (example 25A), this affords 104 mg (90% of theory) of the target compound.

¹H NMR (400 MHz, DMSO-d₆): δ=3.83 (s, 3H), 6.93 (s, 1H), 7.06 (dd, 1H), 7.28 (d, 1H), 7.28-7.37 (m, 1H), 7.38 (d, 1H), 7.53 (dtd, 1H), 7.73 (ddt, 1H), 9.04 (s, 1H), 13.46 (br. s, 1H).

LC-MS (method 12): Rₜ=3.38 min; m/z=392 [M+H]⁺.

### Example 16

### 4-(2-Chlorophenoxy)-6-(2,3-difluorophenyl)nicotinic acid

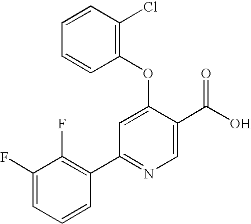

The title compound is prepared, worked up and purified analogously to example 10. Starting from 70 mg (0.19 mmol) of methyl 4-(2-chlorophenoxy)-6-(2,3-difluorophenyl)nicotinate (example 26A), this affords 67 mg (99% of theory) of the target compound.

¹H NMR (400 MHz, DMSO-d₆): δ=6.99 (s, 1H), 7.28-7.45 (m, 3H), 7.45-7.59 (m, 2H), 7.67-7.79 (m, 2H), 9.07 (s, 1H), 13.50 (br. s, 1H).

LC-MS (method 12): Rₜ=3.33 min; m/z=362 [M+H]⁺.

### Example 17

### 4-(2-Chlorophenoxy)-6-(2,4-difluorophenyl)nicotinic acid

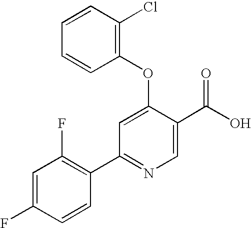

The title compound is prepared and worked up analogously to example 1. Starting from 105 mg (0.28 mmol) of methyl 4-(2-chlorophenoxy)-6-(2,4-difluorophenyl)nicotinate (example 29A), this affords 100 mg (99% of theory) of the target compound.

¹H NMR (400 MHz, DMSO-d₆): δ=6.97 (s, 1H), 7.23 (td, 1H), 7.33 (ddd, 1H), 7.36-7.43 (m, 2H), 7.49 (ddd, 1H), 7.70 (dd, 1H), 8.06 (td, 1H), 9.05 (s, 1H), 13.45 (br. s, 1H).

LC-MS (method 3): Rₜ=3.56 min; m/z=362 [M+H]⁺.

### Example 18

### 4-(2-Chlorophenoxy)-6-(3-chlorophenyl)nicotinic acid

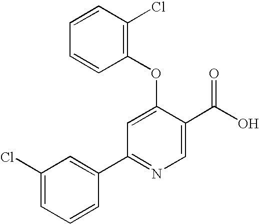

260 μl (0.26 mmol) of a 1M solution of lithium hydroxide in water and also 1.0 ml of water are added to 67 mg (0.17 mmol) of ethyl 4-(2-chlorophenoxy)-6-(3-chlorophenyl)nicotinate (example 31A) in 4 ml of THF, and the mixture is stirred at RT overnight. For work up, the mixture is adjusted to pH 3 with 0.1N hydrochloric acid and partitioned between water and ethyl acetate, and the aqeuous phase is extracted twice more with ethyl acetate. The combined organic phases are dried over sodium sulfate and concentrated. For purification, the residue is separated by preparative HPLC (method 11) to obtain 59 mg (95% of theory) of the target compound.

¹H NMR (400 MHz, DMSO-d₆): δ=7.25-7.37 (m, 2H), 7.27 (s, 1H), 7.45 (td, 1H), 7.48-7.57 (m, 2H), 7.67 (dd, 1H), 7.88 (br. d, 1H), 8.04 (br. s, 1H), 9.04 (s, 1H), 12.8-13.6 (br, 1H).

LC-MS (method 12): Rₜ=3.57 min; m/z=360 [M+H]⁺.

### Example 19

### 4-(2-Chloro-4-trifluoromethoxyphenoxy)-6-(3-chlorophenyl)nicotinic acid

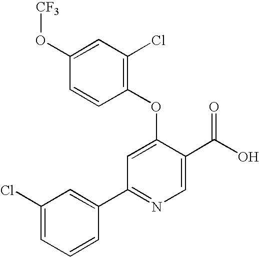

The title compound is prepared, worked up and purified analogously to example 18. Starting from 86 mg (0.18 mmol) of ethyl 4-(2-chloro-4-trifluoromethoxyphenoxy)-6-(3-chlorophenyl)nicotinate (example 32A), this affords 76 mg (94% of theory) of the target compound.

¹H NMR (400 MHz, DMSO-d₆): δ=7.32 (d, 1H), 7.43 (dd, 1H), 7.48-7.58 (m, 2H), 7.57 (s, 1H), 7.82 (d, 1H), 8.01 (dt, 1H), 8.11-8.14 (m, 1H), 9.08 (s, 1H), 13.44 (br. s, 1H).

LC-MS (method 12): Rₜ=3.96 min; m/z=444 [M+H]⁺.

### Example 20

### 6-(3-Chloro-4-fluorophenyl)-4-(2-chlorophenoxy)nicotinic acid

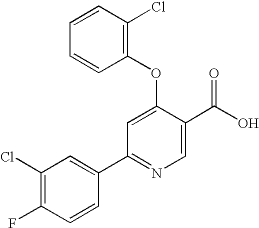

The title compound is prepared, worked up and purified analogously to example 10. Starting from 107 mg (0.26 mmol) of ethyl 6-(3-chloro-4-fluorophenyl)-4-(2-chlorophenoxy)nicotinate (example 33A), this affords 96 mg (96% of theory) of the target compound.

¹H NMR (400 MHz, DMSO-d₆): 7.26 (dd, 1H), 7.32 (s, 1H), 7.33 (td, 1H), 7.43 (td, 1H), 7.51 (t, 1H), 7.66 (dd, 1H), 7.97 (ddd, 1H), 8.23 (dd, 1H), 9.03 (s, 1H), 13.43 (br. s, 1H).

LC-MS (method 1): Rₜ=2.79 min; m/z=378 [M+H]⁺.

### Example 21

### 6-(4-Bromo-2-fluorophenyl)-4-(2-chlorophenoxy)nicotinic acid

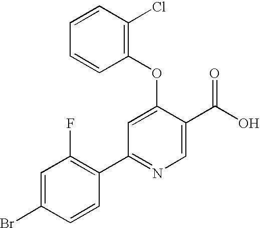

The title compound is prepared, worked up and purified analogously to example 6. Starting from 49 mg (0.11 mmol) of ethyl 6-(4-bromo-2-fluorophenyl)-4-(2-chlorophenoxy)nicotinate (example 35A), this affords 43 mg (94% of theory) of the target compound.

¹H NMR (400 MHz, DMSO-d₆): δ=6.96 (s, 1H), 7.36-7.43 (m, 2H), 7.49 (ddd, 1H), 7.56 (dd, 1H), 7.64 (dd, 1H), 7.70 (d, 1H), 7.96 (t, 1H), 9.06 (s, 1H), 13.48 (br. s, 1H).

LC-MS (method 12): Rₜ=3.70 min; m/z=422 [M+H]⁺.

### B. ASSESSMENT OF THE PHARMACOLOGICAL EFFICACY

The pharmacological action of the inventive compounds can be demonstrated in the following assays:

### 1. Cellular Transactivation Assay:

### a) Test Principle:

A cellular assay is used to identify activators of the peroxisome proliferator-activated receptor alpha (PPAR-alpha).

Since mammalian cells contain different endogenous nuclear receptors which can complicate unambiguous interpretation of the results, an established chimera system is used, in which the ligand binding domain of the human PPARα-receptor is fused to the DNA binding domain of the yeast transcription factors GAL4. The GAL4-PPARα chimera thus formed is co-transfected and expressed stably in CHO cells with a reporter construct.

### b) Cloning:

The GAL4-PPARα expression construct contains the ligand binding domain of PPARα (amino acids 167-468), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, leads to the expression of firefly luciferase (Photinus pyralis) after activation and binding of GAL4-PPARα.

### c) Test Procedure:

The day before the test, CHO (Chinese hamster ovary) cells which stably express the above-described GAL4-PPARα chimera and luciferase reporter gene construct are plated out in 96-hole microtiter plates with 1×10³ cells in medium (Optimem, GIBCO), 2% activated carbon-purified fetal calf serum (Hyclone), 1.35 mM sodium pyruvate (GIBCO), 0.2% sodium bicarbonate (GIBCO), and kept in a cell incubator (air humidity 96%, 5% v/v CO₂, 37° C.). On the day of the test, the substances to be tested are taken up in abovementioned medium, but without addition of calf serum, and added to the cells. After a stimulation time of 6 h, the luciferase activity is measured with the aid of a video camera. The relative light units measured give a sigmoid stimulation curve as a function of the substance concentration. The EC₅₀ values are calculated with the aid of the computer program GraphPad PRISM (Version 3.02).

The table which follows lists the EC₅₀ values of representative example compounds:

TABLE

| Example No. | EC₅₀ [nM] |
|---|---|
| 1 | 59 |
| 2 | 87 |
| 7 | 200 |
| 9 | 235 |
| 2 | 28 |

### 2. Fibrinogen Determination:

To determine the action on the plasma fibrinogen concentration, male Wistar rats or NMRI mice are treated with the substance to be studied by gavage administration or by means of addition to feed for a period of 4-9 days. Under terminal anesthesia, citrate blood is then obtained by heart puncture. The plasma fibrinogen level is determined by the Claus method [A. Claus, Acta Haematol. 17, 237-46 (1957)] by measuring the thrombin time with human fibrinogen as the standard.

3. Test Description for the Discovery of Pharmacologically Active Substances which Increase Apoprotein A1 (ApoA1) and HDL Cholesterol (HDL-C) in the Serum of Transgenic Mice which have been Transfected with the Human ApoA1 Gene (hApoA1) or Lower the Serum Triglycerides (TG):

The substances which are to be examined in vivo for their HDL-C-increasing action are administered orally to male transgenic hApoA1 mice. One day before the start of the experiment, the animals are assigned randomly to groups with the same number of animals, generally n=7-10. Over the entire experiment, the animals have drinking water and feed ad libitum. The substances are administered orally every day for 7 days. For this purpose, the test substances are dissolved in a solution of Solutol HS 15+ethanol+sodium chloride solution (0.9%) in a ratio of 1+1+8 or in a solution of Solutol HS 15+sodium chloride solution (0.9%) in a ratio of 2+8. The dissolved substances are administered in a volume of 10 ml/kg of body weight with a gavage. The control group used is composed of animals which are treated in exactly the same way but receive only the solvent (10 ml/kg of body weight) without test substance.

Before the first substance administration, blood is taken from every mouse by puncturing the retroorbital venous plexus to determine ApoA1, serum cholesterol, HDL-C and serum triglycerides (TG) (zero value). Subsequently, the test substance is administered to the animals for the first time with a gavage. 24 hours after the last substance administration (on the 8th day after the start of treatment), blood is again taken from each animal by puncturing the retroorbital venous plexus to determine the same parameters. The blood samples are centrifuged and, after obtaining the serum, TG, cholesterol, HDL-C and human ApoA1 are determined with a Cobas Integra 400 plus unit (Cobas Integra, from Roche Diagnostics GmbH, Mannheim) using the particular cassettes (TRIGL, CHOL2, HDL-C and APOAT). HDL-C is determined by gel filtration and post-column derivatization with MEGA cholesterol reagent (from Merck KGaA) analogously to the method of Garber et al. [J. Lipid Res. 41, 1020-1026 (2000)].

The action of the test substances on the HDL-C, hApoA1 and TG concentrations is determined by subtracting the measurement from the 1st blood sample (zero value) from the measurement of the 2nd blood sample (after treatment). The differences of all HDL-C, hApoA1 and TG values of one group are averaged and compared to the mean of the differences of the control group. The statistical evaluation is effected with Student t's test after previously checking the variances for homogeneity.

Substances which increase the HDL-C of the animals treated, compared to the control group, in a statistically significant manner (p<0.05) by at least 20%, or lower the TG in a statistically significant manner (p<0.05) by at least 25%, are considered to be pharmacologically active.

### 4. DOCA/Salt Model:

The administration of deoxycorticosterone acetate (DOCA) in combination with a high-salt diet and removal of one kidney induces hypertension in rats, which is characterized by a relatively low renin level. A consequence of this endocrine hypertension (DOCA is a direct precursor of aldosterone), depending on the DOCA concentration selected, is hypertrophy of the heart and further end organ damage, for example to the kidney, which is characterized by features including proteinuria and glomerulosclerosis. In this rat model, it is thus possible to examine test substances for antihypertrophic and end organ-protective action present.

Male Sprague Dawley (SD) rats of about 8 weeks of age (body weight between 250 and 300 grams) are uninephrectomized on the left side. To this end, the rats are anesthetized with 1.5-2% isoflurane in a mixture of 66% N₂O and 33% O₂, and the kidney is removed through a flank section. The later control animals used are so-called sham-operated animals from which no kidney has been removed. Uninephrectomized SD rats received 1% sodium chloride in drinking water and, once per week, a subcutaneous injection of desoxycorticosterone acetate (dissolved in sesame oil; from Sigma) injected between the shoulder blades (high dose: 100 mg/kg/week s.c.; normal dose: 30 mg/kg/week s.c.).

The substances which are to be examined in vivo for their protective action are administered by gavage or via the feed (from Ssniff) or drinking water. One day before the start of the experiment, the animals are randomized and assigned to groups with the same number of animals, generally n=10. Over the entire experiment, drinking water and feed are available to the animals ad libitum. The substances are administered once per day for 4-6 weeks via gavage, feed or drinking water. The placebo group used is animals which have been treated in exactly the same way but receive either only the solvent or the feed or drinking water without test substance.

The action of the test substances is determined by measuring hemodynamic parameters [blood pressure, heart rate, intropy (dp/dt), relaxation time (tau), maximum left-ventricular pressure, left ventricular end-diastolic pressure (LVEDP)], weight determination of heart, kidney and lung, measure of protein excretion and by measuring the gene expression of biomarkers (e.g. ANP, atrial natriuretic peptide, and BNP, brain natriuretic peptide) by means of RT/TaqMan-PCR after RNA isolation from cardiac tissue.

The statistical evaluation is effected with Student t's test after previously checking the variances for homogeneity.

### C. WORKING EXAMPLES FOR PHARMACEUTICAL COMPOSITIONS

The inventive compounds can be converted to pharmaceutical formulations as follows:

### Tablet:

### Composition:

100 mg of the inventive compound, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm

### Production:

The mixture of inventive compounds, lactose and starch is granulated with a 5% solution (m/m) of the PVP in water. After drying, the granule is mixed with the magnesium stearate for 5 minutes. This mixture is pressed with a customary tablet press (see above for format of the tablet). The guide value used for the compression is a pressing force of 15 kN.

### Orally Administrable Suspension:

### Composition:

1000 mg of the inventive compound, 1000 mg of ethanol (96%), 400 mg of Rhodigel® (xanthan gum from FMC, Pennsylvania, USA) and 99 g of water.

10 ml of oral suspension corresponds to a single dose of 100 mg of the inventive compounds.

### Production:

The Rhodigel is suspended in ethanol, and the inventive compound is added to the suspension. The water is added with stirring. The mixture is stirred for approx 6 h until the swelling of the Rhodigel is complete.

### Orally Administrable Solution:

### Composition:

500 mg of the inventive compound, 2.5 g of polysorbate and 97 g of polyethylene glycol 400.20 g of oral solution corresponds to a single dose of 100 mg of the inventive compound.

### Production:

The inventive compound is suspended in the mixture of polyethylene glycol and polysorbate with stirring. The stirring operation is continued up to complete dissolution of the inventive compound.

### I.V. Solution:

The inventive compound is dissolved in a physiologically compatible solvent (e.g. isotonic saline, 5% glucose solution and/or 30% PEG 400 solution) in a concentration below the saturation solubility. The solution is filtered under sterile conditions and filled into sterile and pyrogen-free injection vessels.

1. A compound of the formula (I) 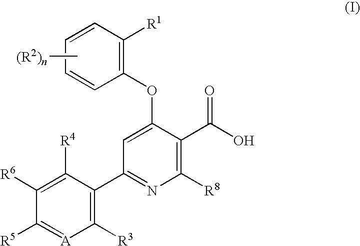
in which
R¹ is halogen, cyano or (C₁-C₄)-alkyl,
R² is a substituent selected from the group of halogen, cyano, (C₁-C₆)-alkyl, (C₁-C₆)-alkoxy and —NR⁹—C(═O)—R¹⁰, in which alkyl and alkoxy may in turn be substituted by hydroxyl, (C₁-C₄)-alkoxy, amino, mono-(C₁-C₄)-alkylamino or di-(C₁-C₄)-alkylamino, or up to pentasubstituted by fluorine, and
R⁹ is hydrogen or (C₁-C₆)-alkyl
and
R¹⁰ is hydrogen, (C₁-C₆)-alkyl or (C₁-C₆)-alkoxy,
n is 0, 1, 2 or 3,
where, in the case that the substituent R² occurs more than once, its definitions may be identical or different,
A is N or C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, (C₁-C₄)-alkyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkyl-amino or di-(C₁-C₄)-alkylamino or up to pentasubstituted by fluorine,
and
R⁸ is hydrogen, methyl or trifluoromethyl,
and the salts, solvates and solvates of the salts thereof.

2. A compound of the formula (I) as claimed in claim 1, in which
R¹ is fluorine, chlorine, bromine, cyano or (C₁-C₄)-alkyl,
R² is a substituent selected from the group of fluorine, chlorine, bromine, cyano, (C₁-C₄)-alkyl and (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
n is 0, 1 or 2,
where, in the case that the substituent R² occurs more than once, its definitions may be the same or different,
A is N or C—R⁷,
R³ is hydrogen or fluorine,
R⁴ is hydrogen, fluorine or methyl,
R⁵ is hydrogen, fluorine, chlorine, cyano, trifluoromethyl, (C₁-C₄)-alkyl, trifluoromethoxy or (C₁-C₄)-alkoxy,
R⁶ and R⁷ are the same or different and are each independently hydrogen, fluorine, chlorine, bromine, cyano, (C₁-C₄)-alkyl or (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
and
R⁸ is hydrogen, methyl or trifluoromethyl,
and the salts, solvates and solvates of the salts thereof.

3. A compound of the formula (I) as claimed in claim 1 in which
R¹ is fluorine, chlorine, bromine, cyano or methyl,
R² is a substituent selected from the group of fluorine, chlorine, bromine, cyano, (C₁-C₄)-alkyl, trifluoromethyl, (C₁-C₄)-alkoxy and trifluoromethoxy,
n is 0, 1 or 2,
where, in the case that the substituent R² occurs more than once, its definitions may be the same or different,
A is C—R⁷,
R³ is hydrogen,
R⁴ is hydrogen or fluorine,
R⁵ is hydrogen, fluorine, chlorine, methyl or trifluoromethyl,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
and
R⁸ is hydrogen,
and the salts, solvates and solvates of the salts thereof.

4. A process for preparing compounds of the formula (I) as defined in claim 1, wherein a compound of the formula (II) 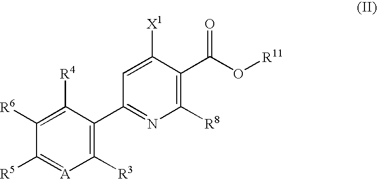
in which A, R³, R⁴, R⁵, R⁶ and R⁸ are each defined as specified in claim 1,
X¹ is a suitable leaving group, for example halogen,
and
R¹¹ is (C₁-C₄)-alkyl,
in an inert solvent in the presence of a base, is reacted with a compound of the formula (III) 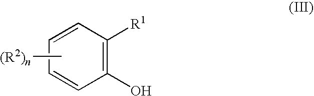
in which R¹, R² and n are each defined as specified in claim 1 to give compounds of the formula (IV) 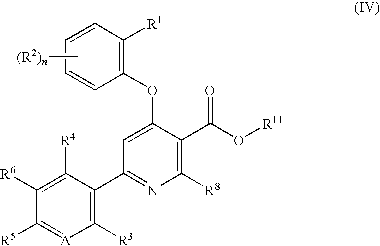
in which A, R¹, R², R³, R⁴, R⁵, R⁶, R⁸, R¹¹ and n are defined as specified above, and these compounds are then converted to the carboxylic acids of the formula (I) by basic or acidic hydrolysis
and the compounds of the formula (I) are optionally reacted with the corresponding (i) solvents and/or (ii) bases or acids to give their solvates, salts and/or solvates of the salts.

5. A process for preparing compounds of the formula (I) as defined in claim 1, wherein a compound of the formula (V) 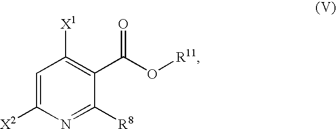
in which R⁸ is as defined in claim 1,
R¹¹ is (C₁-C₄)-alkyl
and
X¹ and X² are the same or different and are each a suitable leaving group, for example halogen, especially chlorine,
is first reacted in an inert solvent in the presence of a base with a compound of the formula (III) 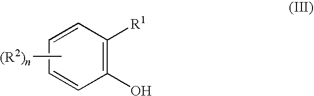
in which R¹, R² and n are each as defined in claim 1
to give compounds of the formula (X) 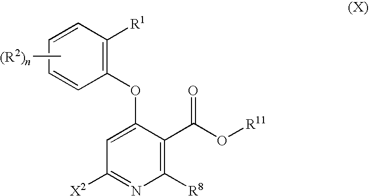
in which R¹, R², R⁸, R¹¹, X² and n are each as defined above,
then this is coupled in an inert solvent in the presence of a suitable transition metal catalyst and optionally of a base to a compound of the formula (VIa) 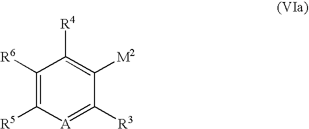
in which A, R³, R⁴, R⁵ and R⁶ are each as defined in claim 1 and
M² is the —B(OH)₂, —ZnHal or —MgHal group, in which
Hal is halogen, especially chlorine, bromine or iodine,
to give compounds of the formula (IV) 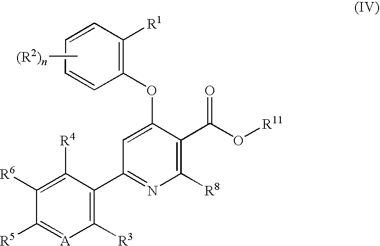
in which A, R¹, R², R³, R⁴, R⁵, R⁶, R⁸, R¹¹ and n are each as defined above, and then converted by basic or acidic hydrolysis to the carboxylic acids of the formula (I),
and the compounds of the formula (I) are optionally reacted with the corresponding (i) solvents and/or (ii) bases or acids to give their solvates, salts and/or solvates of the salts.

6. A compound of the formula (I) as defined in claim 1 for the treatment and/or prophylaxis of diseases.

7. (canceled)

8. A pharmaceutical composition comprising a compound of the formula (I) as defined in claim 1 in combination with an inert, non-toxic, pharmaceutically suitable assistant.

9. The pharmaceutical composition of claim 8, further comprising one or more active ingredients selected from the group consisting of HMG-CoA reductase inhibitors, diuretics, beta-receptor blockers, organic nitrates and NO donors, ACE inhibitors, angiotensin AII antagonists, aldosterone and mineralocorticoid receptor antagonists, vasopressin receptor antagonists, thrombocyte aggregation inhibitors and anticoagulants.

10. The pharmaceutical composition of claim 8 for the treatment and/or prophylaxis of dyslipidemias, arteriosclerosis and heart failure.

11. A method for the treatment and/or prophylaxis of dyslipidemias, arteriosclerosis and heart failure in humans and animals by administering a therapeutically effective amount of at least one compound of the formula (I) as defined in claim 1.

12. A method for the treatment and/or prophylaxis of dyslipidemias, arteriosclerosis and heart failure in humans and animals by administering a therapeutically effective amount of a pharmaceutical composition of claim 8.
